SEQUENCE LISTING (47 sequences)

SEQ ID NO: 1 - DNA, 28 bp, Artificial Sequence
  TN13s Forward (Fw) Primer (5′ - 3′) - for amplification of the UAS promoter
atggcgcgccgcatgcctgcaggtcgga

SEQ ID NO: 2 - DNA, 28 bp, Artificial Sequence
  TN13as Reverse (Rev) Primer (5′ - 3′) - for amplification of the UAS promoter
atttaattaacggggatccggttctctc

SEQ ID NO: 3 - DNA, 30 bp, Artificial Sequence
  TN14s Forward (Fw) Primer (5′ - 3′) - for amplification of the BAR gene
atttaattaaatgagcccagaacgacgccc

SEQ ID NO: 4 - DNA, 31 bp, Artificial Sequence
  TN14as Reverse (Rev) Primer (5′ - 3′) for amplification of the BAR gene
atgcggccgcgatttcggtgacgggcaggac

SEQ ID NO: 5 - DNA, 33 bp, Artificial Sequence
  TN12s Forward (Fw) Primer (5′ - 3′) - for amplification of GAL4VP16
atttaattaaatgaagctcctgtcctccatcga

SEQ ID NO: 6 - DNA, 33 bp, Artificial Sequence
  TN12as Reverse (Rev) Primer (5′ - 3′) for amplification of GAL4VP16
atgcggccgcctacccaccgtactcgtcaattc

SEQ ID NO: 7 - DNA, 30 bp, Artificial Sequence
  TS15s Forward (Fw) Primer (5′ - 3′) - for amplification of the PRPS5a promoter
aggcgcgccgggccataatcgtgagtagat

SEQ ID NO: 8 - DNA, 27 bp, Artificial Sequence
  TS15as Reverse (Rev) Primer (5′ - 3′) - for amplification of the PRPS5a promoter
acgatcgcggctgtggtgagagaaaca

SEQ ID NO: 9 - DNA, 549 bp, Artificial Sequence
  BAR gene amplfied from pGreenII 0229
atgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggtccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtgccgccccgtccggtcctgcccgtcaccgaaatc

SEQ ID NO: 10 - DNA, 188 bp, Artificial Sequence
  UAS promoter
gcatgcctgcaggtcggagtactgtcctccgagcggagtactgtcctccgagcggagtactgtcctccgagcggagtactgtcctccgagcggagtactgtcctccgagcggagactctagaggatcttcgcaagacccttcctctatataaggaagttcatttcatttggagagaaccggatccccg

SEQ ID NO: 11 - DNA, 1688 bp, Arabidopsis thaliana
gggccataatcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaattaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccg

SEQ ID NO: 12 - DNA, 716 bp, Artificial Sequence
  YFP gene
tgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagt

SEQ ID NO: 13 - DNA, 681 bp, Artificial Sequence
  GAL4VP16 construct
tgaagctcctgtcctccatcgagcaggcctgcgacatctgccgcctcaagaagctcaagtgctccaagaagaagccgaagtgcgccaagtgtctgaagaacaactgggagtgtcgctactctcccaaaaccaagcgctccccgctgacccgcgcccacctcaccgaagtggagtcccgcctggagcgcctggagcagctcttcctcctgatcttccctcgagaggacctcgacatgatcctgaaaatggactccctccaggacatcaaagccctgctcaccggcctcttcgtccaggacaacgtgaacaaagacgccgtcaccgaccgcctggcctccgtggagaccgacatgcccctcaccctgcgccagcaccgcatcagcgcgacctcctcctcggaggagagcagcaacaagggccagcgccagttgaccgtctcgacggcccccccgaccgacgtcagcctgggggacgagctccacttagacggcgaggacgtggcgatggcgcatgccgacgcgctagacgatttcgatctggacatgttgggggacggggattccccggggccgggatttaccccccacgactccgccccctacggcgctctggatacggccgacttcgagtttgagcagatgtttaccgatgcccttggaattgacgagtacggtgggtagc

SEQ ID NO: 14 - DNA, 20 bp, Artificial Sequence
  Forward (Fw) Primer (5′ - 3′) targeting the PD2 construct (UAS::BAR_YFP)
tatagggcgaattgggtacc

SEQ ID NO: 15 - DNA, 22 bp, Artificial Sequence
  Reverse (Rev) Primer (5′ - 3′) targeting the PD2 construct (UAS::BAR_YFP)
ggaactggcatgacgtgggttt

SEQ ID NO: 16 - DNA, 22 bp, Artificial Sequence
  Forward (Fw) Primer (5′ - 3′) targeting the PD1 construct (pRPS5a::mGAL4-VP16)
tcgttttctctgccgtctctct

SEQ ID NO: 17 - DNA, 20 bp, Artificial Sequence
  Reverse (Rev) Primer (5′ - 3′) targeting the PD1 construct (pRPS5a::mGAL4-VP16)
cccttgttgctgctctcctc

SEQ ID NO: 18 - DNA, 28 bp, Artificial Sequence
  DT51 Forward (Fw) Primer (5′ - 3′)
caggacatccacctagaaacaagaagac

SEQ ID NO: 19 - DNA, 28 bp, Artificial Sequence
  DT52 Reverse (Rev) Primer (5′ - 3′)
tgaatgtgtgcagatcatggagaaggac

SEQ ID NO: 20 - DNA, 24 bp, Artificial Sequence
  DT53 Forward (Fw) Primer (5′ - 3′)
tgtacctggattcgatagaactgc

SEQ ID NO: 21 - DNA, 24 bp, Artificial Sequence
  DT54 Reverse (Rev) Primer (5′ - 3′)
agaagtccaagcatgtcctatggt

SEQ ID NO: 22 - DNA, 25 bp, Artificial Sequence
  DT55 Forward (Fw) Primer (5′ - 3′)
taagattgctcagaagctgagtgag

SEQ ID NO: 23 - DNA, 25 bp, Artificial Sequence
  DT56 Reverse (Rev) Primer (5′ - 3′)
cgacacacaatgtatctgtttaccc

SEQ ID NO: 24 - DNA, 27 bp, Artificial Sequence
  DT92 Forward (Fw) Primer (5′ - 3′)
acctctaaacgtgattgcaactgtgag

SEQ ID NO: 25 - DNA, 27 bp, Artificial Sequence
  DT93 Reverse (Rev) Primer (5′ - 3′)
tgtgccaacaatctctcatgtacatgg

SEQ ID NO: 26 - DNA, 27 bp, Artificial Sequence
  DT59 Forward (Fw) Primer (5′ - 3′)
tggtcgttgcttaagttgttaagctcg

SEQ ID NO: 27 - DNA, 28 bp, Artificial Sequence
  DT60 Reverse (Rev) Primer (5′ - 3′)
caatacaagacactttctccaatatggg

SEQ ID NO: 28 - DNA, 27 bp, Artificial Sequence
  DT61 Forward (Fw) Primer (5′ - 3′)
tggttcacatgtacgccaagtgttacc

SEQ ID NO: 29 - DNA, 27 bp, Artificial Sequence
  DT89 Reverse (Rev) Primer (5′ - 3′)
cttcccttcattaaccatccctgtgtg

SEQ ID NO: 30 - DNA, 27 bp, Artificial Sequence
  DT116 Forward (Fw) Primer (5′ - 3′)
ttatttccgactttgtctttgccatcg

SEQ ID NO: 31 - DNA, 27 bp, Artificial Sequence
  DT117 Reverse (Rev) Primer (5′ - 3′)
tgctagattatgcctttgatcacaagc

SEQ ID NO: 32 - DNA, 27 bp, Artificial Sequence
  DT65 Forward (Fw) Primer (5′ - 3′)
gctgcaatggtaaacaaggaggaagag

SEQ ID NO: 33 - DNA, 27 bp, Artificial Sequence
  DT66 Reverse (Rev) Primer (5′ - 3′)
cgggatcataatgccgatattcttcgg

SEQ ID NO: 34 - DNA, 27 bp, Artificial Sequence
  DT67 Forward (Fw) Primer (5′ - 3′)
tgtcttcagcatatatgccgattggag

SEQ ID NO: 35 - DNA, 27 bp, Artificial Sequence
  DT68 Reverse (Rev) Primer (5′ - 3′)
caatctcttcaggtgagataatgagcg

SEQ ID NO: 36 - DNA, 27 bp, Artificial Sequence
  DT96 Forward (Fw) Primer (5′ - 3′)
acacgcaagagagaattggatgatacc

SEQ ID NO: 37 - DNA, 27 bp, Artificial Sequence
  DT97 Reverse (Rev) Primer (5′ - 3′)
agcagaatctcaatctcgatggttgtg

SEQ ID NO: 38 - DNA, 27 bp, Artificial Sequence
  DT71 Forward (Fw) Primer (5′ - 3′)
tctcttgctctcttctctctcacacag

SEQ ID NO: 39 - DNA, 27 bp, Artificial Sequence
  DT72 Reverse (Rev) Primer (5′ - 3′)
acctcttgtggagtatcaagccaagtg

SEQ ID NO: 40 - DNA, 27 bp, Artificial Sequence
  DT98 Forward (Fw) Primer (5′ - 3′)
tacagagtccaagtcgttgtcatgctc

SEQ ID NO: 41 - DNA, 27 bp, Artificial Sequence
  DT99 Reverse (Rev) Primer (5′ - 3′)
tgcgtgactgctatatcctaaaggagg

SEQ ID NO: 42 - DNA, 25 bp, Artificial Sequence
  DT77 Forward (Fw) Primer (5′ - 3′)
caacgagaagttggattgtgaggtg

SEQ ID NO: 43 - DNA, 25 bp, Artificial Sequence
  DT78 Reverse (Rev) Primer (5′ - 3′)
ggatgaaggagcatatggagatagc

SEQ ID NO: 44 - DNA, 27 bp, Artificial Sequence
  DT79 Forward (Fw) Primer (5′ - 3′)
gtattcacgaccatcgtagctgtccac

SEQ ID NO: 45 - DNA, 27 bp, Artificial Sequence
  DT80 Reverse (Rev) Primer (5′ - 3′)
aactcgcctctgaagctggtcaatgag

SEQ ID NO: 46 - DNA, 27 bp, Artificial Sequence
  DT81 Forward (Fw) Primer (5′ - 3′)
aacagtaatggtccccattcacggtgg

SEQ ID NO: 47 - DNA, 27 bp, Artificial Sequence
  DT82 Reverse (Rev) Primer (5′ - 3′)
ccaattagaccttgaaggagcaggacc

(19) United States
(12) Patent Application Publication
(10) Pub. No.: US 2019/0159417 A1
(43) Pub. Date: May 30, 2019

(54) METHOD OF GENERATING POLYPLOID PLANTS

(71) Applicant: University of Bremen, Bremen (DE)

(72) Inventor: Rita GROß-HARDT, Bremen (DE)

(21) Appl. No.: 16/318,830

(22) PCT Filed: Jul. 18, 2017
(86) PCT No.: PCT/EP2017/068133

§ 371 Date: Jan. 18, 2019

(30) Foreign Application Priority Data
Jul. 18, 2016 (EP) 16179967.1

(51) Int. Cl. A01H 1/08; A01H 1/04; A01H 1/02

(52) U.S. Cl. CPC A01H 1/08; A01H 1/02; A01H 1/04

(57) ABSTRACT

Polyploid plants obtainable by crossing of more than two parent plants within one generation as well as a novel high-throughput polypaternal breeding design (HIPOD) method allowing such polypaternal crossing are provided. In particular, triparental plants are described.

### FIELD OF THE INVENTION

The present invention relates to hybrid plants, in particular polyploid plants obtainable via a novel method crossing of more than two parent plants as well as to organs, tissue, parts, cells, seeds or offspring of such plants. Furthermore the present invention relates to a marker-assisted method for generating such plants.

### BACKGROUND OF THE INVENTION

These days, the agricultural sector faces tremendous challenges in providing sufficient food for the constantly growing world population which comes along with a reduced availability of arable land. Simultaneously, the need of agricultural crops has increased due to their use for the generation of energy and as substitutes for chemical compounds. Thus, due to the growing demand for agricultural goods, the key task of plant breeding is the increase of the crop yield as well as the improvement of certain plant characteristics such as heat or pest resistance. Nowadays, also the supply with convenient food, such as seed reduced eggplants or melons becomes more and more important. The improvement of plants requires the development of fast and efficient breeding procedures to render it economic.

Hitherto, the commonly used tool in plant breeding is hybridization of two parental lines which are preferably genetically distinct as it allows the combination of different germ plasms conferring agriculturally valuable traits in a single plant by intercrossing. However, hybridization of two different parental lines does not always result in a positive outcome but might lead to hybrid incompatibility resulting in offspring that is impaired in its growth and productivity, which is sterile or even inviable. In many cases, this incompatibility is mounted in the endosperm, which is the nurturing tissue for the developing embryo in the seed and forms after fertilization of the central cell.

Furthermore, the ability of plants to tolerate the presence of supernumerary genomic copies is used to improve the crop yield of plants, to establish novel variations and morphologies or to improve the genetic diversity of plants to allow them to efficiently adapt to changing environments. These supernumerary genomic copies can arise spontaneously in nature by several mechanisms, including meiotic or mitotic failures, fusion of unreduced (2n) gametes or somatic doublings and can be used for breeding processes. For example, the domestic common wheat was bred to contain six sets of chromosomes, i.e. it is hexaploid which results in a higher crop yield in comparison to the wild wheat which is diploid (2n). The selective generation and use of plants which produce unreduced microspores and/or pollen grains is also a well-known mechanisms in plant breeding; see, e.g., international application WO 2010/149322 A1.

In many plants, the introgression of supernumerary paternal copies by diploid sperm is associated with a postzygotic hybrid incompatibility reaction. Notably, this so called triploid block is mounted in the endosperm (Kohler et al., Tends Genetic. 26 (2010), 142-148) and can lead to mortality, reduced viability or reduced fertility of the hybrid. This latter phenomena is used, e.g., for the generation of seed reduced triploid plants, e.g. eggplants via crossing of a tetraploid with a diploid plant; see, e.g., international application WO 2009/095266. However, the offspring is still derived from two parent plants, i.e. it is biparental and to establish and combine favorable genes of different plant sources in the offspring, still multiple breeding cycles are necessary and there is no possibility that at the end the desired genetic information is equally distributed. On the other hand, the approach of tailor-made plants by genetically engineering, i.e. transgenic plants may not always be possible since many traits in crops are polygenic and dependent on the genomic background of the parent plant.

Thus, there is a need for high-performance plants, in particular hybrid and polyploid plants, respectively, which are suitable in breeding programs as well as for a method of obtaining such plants that can be performed in a time and cost effective manner. The above mentioned technical problems are solved by the embodiments characterized in the claims, which are described further below and illustrated in the Examples and Figures.

### SUMMARY OF THE INVENTION

The present invention generally relates to polyploid plants obtainable by crossing of more than two parent plants (polypaternal crossing) within one generation as well as to a novel marker-assisted system allowing such polypaternal crossing. More specifically, the present invention relates to polyparental, preferably triparental plants and their use in agriculture and agronomics. The polyparental plants of the present invention which arise from fusion of at least three germ cells and hence contain the full genome of at least three genetically distinct parents are to be distinguished from plants simply derived from generic triparentage, i.e. from plants obtained by successive hybridization leading to the segregation of genetic material from more than two parents. The origin of plants from more than two ancestral plants is known as hybridization of multiple parents over an evolutionary timescale. In this case, the segregation of genetic material from more than two parents is, however, easily explained by successive crossings: A×B=C; C×D=E. This successive hybridization mode neither involves the fusion of three germ cells, nor does any of the offspring have three biological parents. The poly-, e.g. triparental origin of the polyploid plants of the present invention is to be distinguished from that generic triparentage, since in accordance with the present invention the triparental plant arises from fusion of three germ cells and hence contains the full genome of three genetically distinct parents: A+B+C=F.

In order to enable the provision of such triparental plants, in accordance with the present invention, a novel marker-assisted system has been established coined “High-throughput polypaternal breeding design (HIPOD) method” allowing the generation of polyploid plants via crossing of more than two parent plants within one generation and the generation of marker-free polyploid plants in the next generation. Thus, the present invention provides polyploid plants that have been generated via crossing of one female parent plant with at least two male parent plants as well as to organs, tissue, parts, cells, seeds or offspring of the plants, wherein the male parent plants can be genetically identical or distinct.

Although it is commonly known that several plants tolerate the presence of supernumerary genomic copies, plants nevertheless evolved polyspermy barriers that hinder the fusion of more than two gametes since in most eukaryotes polyspermy is lethal or results in reduced fertility or even sterility due to the genomic imbalance; see supra. Polyspermy barriers are implemented at different levels in the reproductive process and a common mechanism for polyspermy barriers found in animals and plants are egg cell blocks, which are mounted after gamete fusion and impose chemical or physical barriers to reduce the risk of further sperm entry (Iwao, Reproduction 144 (2012), 11-22; Scott et al., Mol. Plant. 1 (2008), 611-619; Jaffe, Proc. Natl. Acad. Sci. USA 88 (1991), 9883-9887 (1991); Tsaadon et al., Mol. Cell. Endocrinol. 252 (2006), 107-114; Spielman and Scott, Sexual Plant Reproduction 21 (2008), 53-65). So far, in vitro studies on rice have been performed in which two sperm cells of one rice plant have been electrically fused with the egg cell of another rice plant in order to investigate pathways which may contribute to the formation of autopolyploids (Toda et al., Plant Physiol. 171 (2016), 206-214). However, this approach was not considered for the generation of triploid plants with different male parents or implantation in plant breeding in general, especially since this approach uses sperm cells derived from one single plant still leading to biparental offspring and not to polyparental plants as described in the present invention. This method does not integrate naturally occurring in-planta mechanisms for polyspermy, but is based on a purely synthetic in vitro approach. Therefore, in one embodiment plants are specifically excluded from the present invention, which have been generated in vitro, e.g. by electrofusion such as the rice plants described in Toda et al. (2016).

In contrast, as demonstrated in the appended Examples, the method of the present invention enables crossing of three parental lines including at least two different male parental lines and gives rise to viable offspring which can be used in further breeding programs. In particular, the method of the present invention encompasses labeling the male parent plants each with a component of a selectable marker, crossing the male parent plants with a female parent plant and selecting the offspring for the presence of a selectable marker, which preferably confers herbicide resistance and/or fluorescence when combined in one single plant. Alternatively, the method of the present invention encompasses labeling the male parent plants each with a different selectable marker, crossing the male parent plants with a female parent plant and selecting the offspring for the presence of the two different selectable markers, which preferably confer herbicide and antibiotic resistance, respectively, potentially combined with fluorescence when combined in one single plant. Advantageously, since the F1 offspring of the method of the present invention is heterozygous for the selectable marker gene(s) but “homozygous” as regards its polyploidy, i.e. triparental selfing or crossing of the F1 generation inter alia results in marker gene-free F2 plants devoid of any DNA which is foreign to the plant. Accordingly, also the provision of marker-free polyploid plants in the following generation is encompassed by the present invention. Furthermore, the method of the present invention can be used to successfully cross at least one male parent plant which is incompatible with the female parent plant. Thus, the method of the present invention is suitable to overcome hybrid incompatibility or even species barriers in angiosperms via providing sperm from a compatible and an incompatible crossing partner.

### BRIEF DESCRIPTION OF THE FIGURES

The embodiments of the present invention are described with reference to the attached Figures and sequences.

FIG. 1: Establishment of a high-throughput polypaternal breeding design (HIPOD) method for the generation of polyparental, preferably triparental polyploid plants. (A) The HIPOD method of the present invention comprises the provision of at least three parental plants, wherein at least one first and one second male parent plant comprises a component of a selectable marker system, for example the synthetic transcription factor mGAL4 driven by the ubiquitous NOS or RPS5a promoter (first component, grey), which transactivates the UAS promoter driving an herbicide conferring YFP-tagged BAR gene (second component, black), which when combined through fusion of the sperm cells and the egg cell give rise to BASTA resistance. Of course, as described in the description of the invention also more than two components and male parent plants may be used for complementation of the selectable phenotype and thus for example tetraparental, tetra- or octaploid plants in the offspring. Alternatively, as also outlined in the description, the at least two male parent plants may each comprise a different selectable marker, for example a herbicide and antibiotic conferring resistance gene, respectively, which when combined through fusion of the sperm cells and the egg cell gives rise to plants resistant to the at least two selectable markers. (B) Pollen from pollen donor 1 (PD1) and pollen donor 2 (PD2) (grey and black, respectively) harboring the individual components of the two component system are applied to the stigma of a gynoecium. Fusion of two sperm cells from two different fathers (grey and black, respectively) with the egg cell, but not monospermy, enables transactivation resulting in herbicide resistant F1 plants. (C) Fluorescence microscopy of protoplasts transformed with pRPS5a::mGAL4-VP16 (left), pUAS::BASTA_YFP (middle) or co-transformed with pRPS5a::mGAL4-VP6 and pUAS::BASTA_YFP (right). (D) herbicide-treated F1 plants resulting from selfing of pRPS5a::mGAL4-VP16/+(left), selfing of pUAS::BASTA_YFP/+(middle) and a cross between pRPS5a::mGAL4-VP16/− and pUAS::BASTA_YFP/− (right). (E) YFP fluorescence in sepals of F1 plants resulting from selfing of pRPS5a::mGAL4-VP16/+(left), selfing of pUAS::BASTA_YFP/+(middle) and a cross between pRPS5a::mGAL4-VP16/− and pUAS::BASTA_YFP/− (right). Scale bars 15 μm (C), 0.2 cm (D), and 50 μm (E).

FIG. 2: Generation of plants with three parents (HIPOD). (A) Herbicide treated offspring of diploid wild-type plants (left) and plants generated via the HIPOD method of the present invention (right). (B) YFP fluorescence of diploid wild-type plants (left) and plants generated via the HIPOD method (right). (C) Multiplex PCR targeting pUAS::BASTA_YFP (black) and pRPS5a::mGAL4-VP6 (grey) in (1) herbicide-resistant plants generated via HIPOD, (2) pUAS::BASTA_YFP/+, (3) pRPS5a::mGAL4-VP16/+, (4) pUAS::BASTA_YFP/−, pRPS5a::mGAL4-VP16/−, (5) water control, (6) wild-type control. The crossing scheme resulting in the F1 plants analyzed is indicated in the cartoon. (D) Flow cytometric analysis of diploid wild-type plant (upper panel), triploid wild-type plant (middle panel) and an herbicide-resistant plant generated via HIPOD (lower panel). (E) DAPI-stained chromosome spreads of diploid wild-type plant (left) and plant generated via HIPOD (right). Scale bars 0.4 cm (A), 100 μm (B), and 5 μm (C)

FIG. 3: Polyspermy gives rise to viable triparental plants. (A) Growth height comparison between biparental diploid plants (2n BP), biparental triploid plants recovered from a 2n×4n cross (3n BP), and an herbicide resistant triploid triparental plant (3n TP). (B), (C) inflorescence and flower of 2n BP, 3n BP, and 3n TP plants. (D) Growth height of 2n BP (n=19), 3n BP (n=15), and 3n TP plants (n=7). (E) Petal size of 2n BP (n=68), 3n BP (n=101), and 3n TP plants (n=104). (F) Sepal size of 2n BP (n=89), 3n BP (n=58, and 3n TP plants (n=107). (G) Size of petal epidermis cells in 2n BP (n=24), 3n BP (n=112), and 3n TP plants (n=93). (H), (I) Silique analysis 2n BP (n=19), 3n BP (n=19), and 3n TP (n=39) plants showing fertile ovules (black arrows), sterile ovules (white arrowhead) and aborted ovules (white arrow). (J) Petal epidermis cells of 2n BP, 3n BP, and 3n TP. (K) Seed morphology of 2n BP, 3n BP, and 3n TP. Scale bars 2 cm (a); 1 mm (b, c); 0.5 mm (h); 5 μm (j); 0.5 mm (k). Plot (i) show mean±s.d. P values (***P<0.001) report significance by t-test. Box-plots show median, quartiles, maximum and minimum, outliers: Z>+/−3.

FIG. 4: Polyspermy-induced hybridization of three accessions. (A) Herbicide resistant plant generated via crossing of three accessions (Col-0, C24 pRPS5a::mGAL4-VP16/+ and Ler pUAS::BASTA_YFP/+) (3n TP) using HIPOD. (B) YFP florescence analysis of diploid biparental plant (2n BP) and triploid triparental plant (3n TP). (C) Flow cytometry analysis of 2n biparental (2n BP), 3n biparental (3n BP), and 3n TP. (D) Analysis of accession-characteristic RFLPs on different chromosomes (Chr.) in (1) 3n TP, (2) Col-0 (light grey), (3) Ler (black), and (4) C24 (grey). The crossing scheme resulting in the F1 plants analyzed is indicated in the cartoon. Scale bar: 1 cm (A); 100 μm (B).

FIG. 5: Bypassing hybrid incompatibilities using polyspermy. The method of the present invention allows the crossing of at least two male parent plants with one female parent plant, wherein one of the male parent plants is incompatible with the female parent plant. In particular, first a compatible crossing partner is applied and the respective fertilization will supply both, egg and central cell with compatible sperm. In addition, or slightly later, pollen of a second, non-compatible partner is applied to the stigma. The genetic material of the incompatible crossing partner will be provided to the egg cell, but not to the central cell and the endosperm-triggered hybrid incompatibility is bypassed. Thus, polyspermy in the egg cell will allow for transmission of incompatible sperm while bypassing the endosperm-triggered hybrid incompatibility. Left panel, compatible cross; middle panel, incompatible cross; right panel, two paternal cross to bypass hybridization barriers in the endosperm. Compatible sperm cells (grey) and incompatible sperm cells (black).

### DEFINITIONS

Initially, some of the terms used throughout the specification are defined in the following.

The term “crossed” or “cross” means the fusion of gametes via pollination to produce a progeny and encompasses both sexual crosses (the pollination of one plant by another) and selfing (self-pollination, e.g., when the pollen and ovule are from the same plant individual). The term “crossing” refers to the act of fusing gametes via pollination to produce a progeny. Thus, the expression “crossing of one female parent plant with more than one male parent plants” is used interchangeably with the expression “fertilization of one egg cell with more than two sperm cells”. Of course, in angiosperms “crossing” also includes the fertilization of the endosperm with the sperm cell from male parent plant, preferably from the first male parent plant in case of polypaternal crossings.

The term “cultivar” as used throughout the present description refers to a plant or group of plants selected and bred by man for desirable characteristics such as size, color, yield, disease resistance, taste, etc. that can be maintained by propagation. Thus, cultivars are distinct, uniform and stable. Cultivars are subgroups within the same species. For uncultivated plants (wild flora) the term “variety” is accordingly used.

“Flowering plants” and “angiosperms” as used in the present description are seed-producing plants (spermatophytes) and are distinguished from other seed-producing plants (gymnosperms) by several characteristics including the presence of flowering organs, stamens with two pairs of pollen sacs, reduced male and female gametophytes, closed carpel enclosing the ovules and endosperm formation, wherein the endosperm is a highly nutritive tissue inside the seeds of the flowering plant and provides nutrition to the embryo.

In the context of encoding sequences, promoters, and other genetic elements, the term “heterologous” indicates that the element is derived from a genotypically distinct entity from that of the rest of the entity to which it is being compared.

The term “heterozygous” means a genetic condition of a given individual wherein different alleles reside at corresponding loci on homologous chromosomes. The term “homozygous” means a genetic condition wherein identical alleles reside at corresponding loci on homologous chromosomes.

The term “hybridization” in the context of plant breeding can be used interchangeable with “hybrid cross” and means a cross between two genetically distinct parent plants produced by crossing plants of different lines or breeds or species, including but not limited to the cross between two inbred lines (e.g., a genetically heterozygous or mostly heterozygous individual) to produce a hybrid plant (offspring).

The term “hybrid incompatibility” is commonly used as the collective for hybrid inviability and sterility. This incompatibility can be mounted at different levels of the reproduction process. One can distinguish between hybrid incompatibility which occurs due to differences in the ploidy status of the crossing partners and hybrid incompatibility which is “intrinsic” meaning that it occurs in the absence of ploidy differences or apparent ecological factors isolating lineages. The latter one is thought to be due to negative epistasis between newly derived alleles that have arisen in isolation but are dysfunctional against the genetic background of the other diverged lineage. For example, hybrid seed inviability (abortion) is a common early interspecific barrier among many plant groups and has frequently been attributed to species-specific changes in the endosperm.

In context of the present application, the term “offspring” plant refers to any plant resulting as progeny from a sexual reproduction from one or more parent plants or descendants thereof. For instance, an offspring plant may be obtained by crossing two or more parent plants and include selfings as well as the F1 or F2 or still further generations.

Genetic elements are said to be “operatively linked” if they are in a structural relationship permitting them to operate in a manner according to their expected function. For instance, if a promotor helps to initiate transcription of the coding sequence, the coding sequence can be referred to as operatively linked to (or under control of) the promoter. There may be intervening sequences between the promoter and coding region so long as this functional relationship is maintained.

Unless otherwise stated, a “plant” of the present invention is any plant at any stage of development, preferably a seed plant including dicotyledons and monocotyledons, preferably a flowering plant. “Plant organs” mean for example leaves, stem, roots, root tips, buds, meristems, embryos, anthers, ovules, seeds, flowers or fruits.

The term “plant part” refers to a part of a plant including but not limited to single cells and cell tissues such as plant cells that are intact in plants, cell clumps and tissue cultures from which plants can be regenerated. Examples of plant parts include, but are not limited to, single cells and tissues from pollen, ovules, leaves, embryos, roots, root tips, anthers, flowers, fruits, stems shoots, and seeds; as well as pollen, ovules, leaves, embryos, roots, root tips, anthers, flowers, inflorescence, fruits, stems, shoots, scions, rootstocks, seeds, protoplasts, calli, and the like. The terms “plant cells” or “cells of a plant” are used to describe for example isolated cells containing a cell wall or aggregates thereof or protoplasts.

“Polyploidy” defines a status in which more than two sets of chromosomes are present in a cell, whereas “triploid” plant cells possess three sets of chromosomes, “tetraploid” plant cells four sets, “pentaploids” five sets, “hexaploids” six sets, “heptaploids” seven sets, “octaploids” eight sets, “nonaploids” nine sets, “decaploids” ten sets, “undecaploids” eleven sets and “dodecaploids” twelve sets of chromosomes.

Particular gene sequences referred to as “promoters”, like the NOS promoter, the RPS5a promoter or the UAS promoter, are polynucleotide sequences derived from the gene referred to that promotes transcription of an operatively linked gene expression product. It is recognized that various portions of the upstream and intron untranslated gene sequence may in some instance contribute to promoter activity via proving the binding domain for the RNA-polymerase and via initiating transcription of DNA. The promoter contains further elements functioning as regulator of the gene expression (e.g. cis-regulatory elements). The promoter may be based on the gene sequence of any species having the gene, unless explicitly restricted, and may incorporate any additions, substitutions or deletions desirable, as long as the ability to promote transcription in the target tissue is maintained.

For the purpose of this description the term “polyspermy” refers to the fertilization of one egg cell with more than one sperm cell that may be derived from different pollen tubes and is not to be confused with “double fertilization” which is a commonly known mechanism in angiosperms comprising the fertilization of not only the egg cell but also the egg-adjoining central cell, which comprises the two polar nuclei, to generate the embryo nourishing tissue, i.e. the endosperm which is usually triploid. The two sperm cells necessary for this double fertilization are delivered by a single pollen tube. Furthermore, in the sense of the present invention, polyspermy it is not to be confused with the “physiological polyspermy”, in which the entry of several sperm cells into the egg cell is permitted but only one sperm nucleus participates in the formation of a zygote nucleus, while the other sperm nuclei undergo degeneration.

A “transgenic plant” refers to a plant stably transfected with at least one polynucleotide, preferably a heterologous polynucleotide. Preferably, the polynucleotide is stably integrated meaning that the integrated polynucleotide is stably maintained in the plant, is expressed and also stably passed to the offspring. The stably integration of a polynucleotide into the genome of a plant includes the integration into the genome of the previous parent generation as well, wherein the polynucleotide is stably inherited.

The present invention relates to the embodiments characterized in items [1] to [19] and illustrated in the Examples.
[1] Polyploid plant obtainable by crossing of a female parent plant with at least two male parent plants.

Since flowering plants are commonly known to reproduce sexually and thus carry the genetic material from a male and a female parent plant, and since it is known that plants exhibit polyspermy barriers preventing the egg cell to be fertilized with more than one sperm cell due to incompatibility reactions that might occur otherwise, the generation of a polyploid plant having more than two parents has not been thought about before. As described in the present invention, the inventors developed a high-throughput polypaternal breeding design (HIPOD) method for the generation of polyploid plants having more than two parents i.e. polyparental plants that have been generated via fertilization of one egg cell with more than two sperm cells with is referred to as polyspermy. Thus, the present invention provides a polyploid plant characterized in that it is obtained via crossing of one female parent plant with more than one male parent plant as well as the polyploid seed which is obtained by this crossing and from which the plant develops. The male parent plants can be either genetically distinct or genetically identical. In the following, when referred to the plant of the present invention, it encompasses, unless otherwise stated, the seed from which the plant develops as well.
[2] The polyploid plant of [1], which is a triparental plant.

In one embodiment, the polyploid plant of the present invention is obtained via crossing of one female plant with two, three, four, five, six, seven, eight, nine or ten male parents plants. Preferably, said polyploid plant is obtained via crossing of three parent plants, i.e. via crossing of one female parent plant with two male parent plants, also referred to as triparental.
[3] The polyploid plant of [1] or [2], wherein the parent plants are diploid or tetraploid and/or the polyploid plant is triploid or hexaploid.

In one embodiment, the polyploid plant of the present invention comprises three, four, five, six, seven, eight, nine, ten, eleven or twelve sets of chromosomes, i.e. it is triploid, tetraploid, pentaploid, hexaploid, heptaploid, octaploid, nonaploid, decaploid, undecaploid or dodecaploid and has been obtained by crossing of one female plant with two, three, four, five, six, seven, eight, nine or ten male parents plants, wherein the crossing can occur between parent plants with different ploidy, i.e. they can be haploid, diploid, triploid, tetraploid, pentaploid, hexaploid, heptaploid, octaploid, nonaploid, decaploid, undecaploid or dodecaploid. Preferably, the polyploid plant of the present invention is triploid or hexaploid and has been obtained via crossing of three diploid or tetraploid parent plants, i.e. by one diploid or tetraploid female parent plant and two diploid or tetraploid male parent plants.

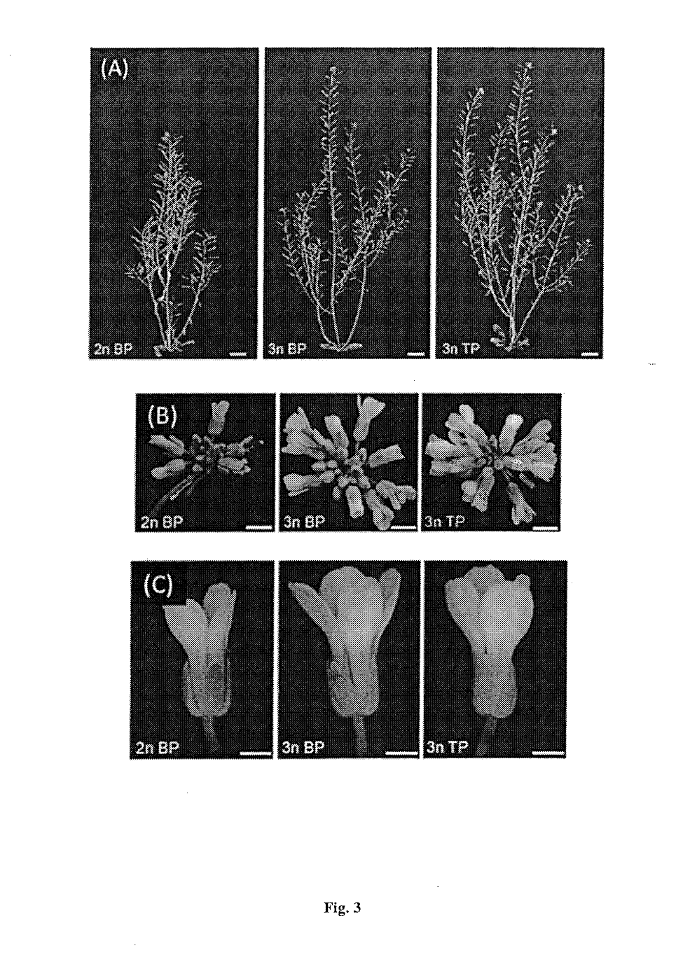

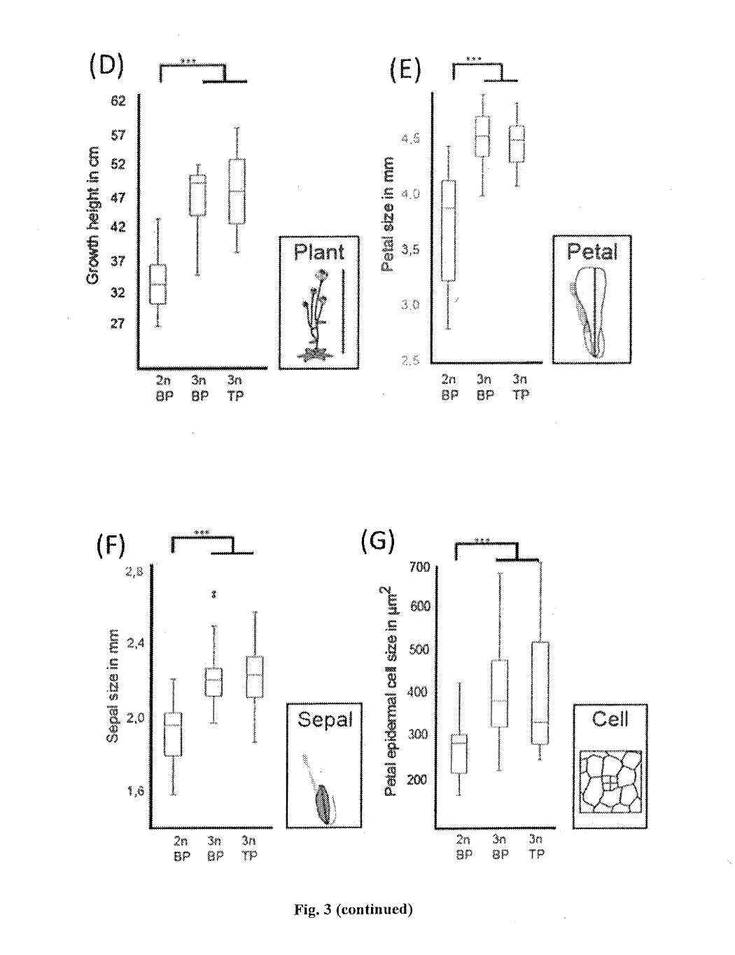

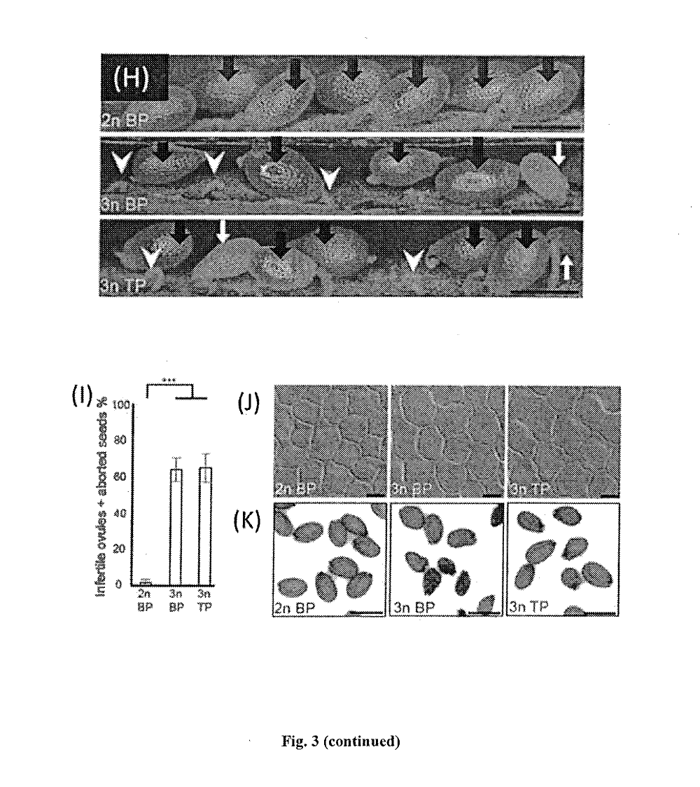

The polyploid plant of the present invention is furthermore viable. In experiments regarding phenotypic characterization of triploid Arabidopsis plants that have been generated via crossing of one diploid female parent plant with two diploid male parent plants it has been shown that the overall plant size of triparental plants was increased 1.5 fold and the plants gave rise to bigger inflorescences and flowers when compared to diploid biparental plants; see FIG. 3 (A)-(D). In addition, it was found that triparental plants produced significantly larger organs in comparison to diploid plants. Furthermore, the petal and sepal size were increased by 20.7% and 17.0% respectively; see FIGS. 3 (E) and (F). In summary, the polyploid plant of the present invention exhibits an increase in the overall plant size as well as an increase in the size of its inflorescences, flowers, organs, petals and sepals when compared to diploid biparental plants.

An inherent consequence of meiosis in triploid plants is aneuploidy, i.e. an imbalance in chromosome number. In fact, it has been observed that fertility in triparental triploid plants, similarly to biparental triploids is substantially reduced accompanied by the segregation of shriveled and malformed ovules and seeds; FIGS. 3 (H), (I) and (K). Thus, the triparental plants of the present invention can also be used for the breeding of seed-reduced fruits, e.g. seedless melons. However, in the further generations, the triploid plants segregate in diploid, triploid and tetraploid plants, wherein the diploid and tetraploid plants do not suffer from aneuploidy-induced sterility.
[4] The polyploid plant of any one of [1] to [3], wherein at least the two male parent plants are genetically distinct, preferably wherein they belong to different cultivars or varieties.

In a further embodiment, a polyploid plant of the present invention is provided, which is obtained by crossing of one female parent plant with at least two male parent plants, wherein the two male parent plants are genetically distinct, preferably wherein they belong to different cultivars or varieties. Thus, in case of wild flora, although the different varieties belong to the same species and therefore can be bred with each other, in general this would not occur naturally since usually they either originate from different areas; i.e. they are geographically isolated and/or they are reproductively isolated. For plant breeding, this opens new possibilities to increase the genetic variety in cultured plants within one generation and due to the fact that the chromosomes of all three partners are equally represented in the polyparental polyploid plant, which would not be possible in a traditional two parent cross as this requires two generations, the traits and protein products are balanced resulting in a better predictability of the outcome of this breeding process.
[5] The polyploid plant of any one of [1] to [4], wherein at least one of the male parent plant is incompatible with the female parent plant.

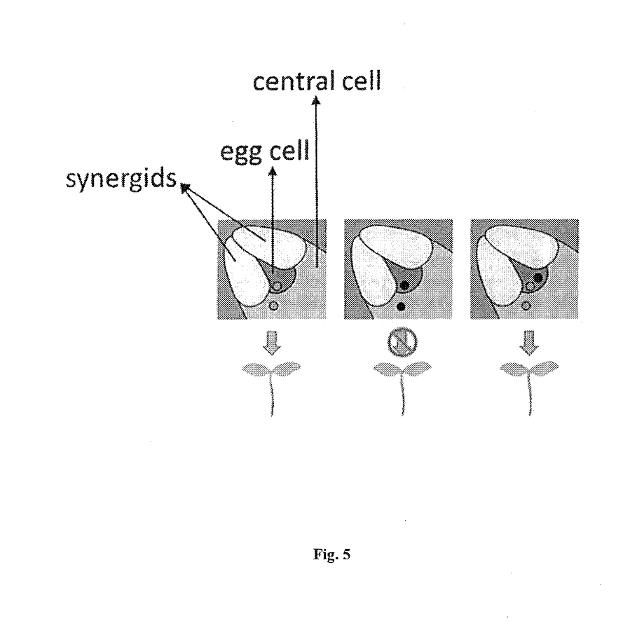

The method of the present invention even allows the crossing of incompatible cultivars or varieties or even different species. Since the recent doctrine teaches that hybrid incompatibility is often mounted in the endosperm, either due to the presence of two paternal genomic copies which could occur e.g. during fertilization with diploid sperm cells or due to genetic incompatibilities between specific genes of the crossing partners, without wishing to be bound to theory it is well conceivable that polyspermy induced triploid embryos develop in seeds where the endosperm did not receive an extra paternal copy, which could bypass the triploid block. This principle is illustrated in FIG. 5 and described further below.

Thus, in one embodiment of the present invention, the polyploid plant of the present invention is obtained by crossing of one female parent plant with at least two male parent plants, wherein at least one of the male parent plants is incompatible with the female parent plant.
[6] The polyploid plant of any one of [1] to [5], which is a flowering plant.

Experiments for enabling the triparental crosses have been performed with Arabidopsis thaliana. Since Arabidopsis thaliana is well known in the art to be a model plant for higher plants, especially for angiosperms, i.e. flowering plants, the ability to generate polyparental polyploid Arabidopsis plants can be transferred to other angiosperms. Thus, according to one embodiment, the present invention comprises, next to Arabidopsis, polyploid angiosperms in general.
[7] The polyploid plant of any one of [1] to [6], which is a dicotyledonous or monocotyledonous plant.

In a preferred embodiment, the present invention relates to in polyploid dicotyledons and monocotyledons that have been obtained via crossing of more than two parent plants. More particularly, the present invention encompasses crop plants and cultured plants, wherein the first ones include cultivated plants as well as uncultivated plants (wild flora) and encompass all plants that are directly or indirectly used by man, e.g. as food, medicine, luxury food, as feed for livestock or wood supplier and wherein the latter ones, i.e. the cultivated plants refer to a plant which is grown and bred by man and used as crop plant or ornamental plant. These cultured plants include amongst others food crops, industrial crops (e.g. fiber crops), feed crops and ornamental plants. Important characteristics of these cultured plants are amongst others the increase in size of the plant, in particular of the used plant organs, the loss of bitter compounds, pest resistance and/or a high content of nutrients. More preferably, in one embodiment, the present invention relates to plants which are selected from the plants mentioned below.
[8] The polyploid plant of any one of [1] to [7], which is selected from the group consisting of Zea, Solanum, Triticum, Triticale, Helianthus, Secale, Hordeum, Brassica, Brachypodium, Glycine, Gossypium, Sorghum, Saccharum, Setaria, Aegilops, Oryza, Daucus, Eucalyptus, Erythranthe, Genlisea, Musa, Avena, Nicotiana, Coffea, Vitis, Cucumis, Morus, Crucihimalaya, Cardamine, Lepidium, Capsella, Olmarabidopsis, Arabis, Raphanus, Eruca, Citrus, Jatropha, Populus, Beta and Manihot, preferably, wherein the plant is selected from the group consisting of the species Zea mays, Solanum tuberosum, Triticum aestivum, Triticum durum, Triticum spelta, Helianthus annuus, Secale cereale, Hordeum vulgare, Hordeum bulbosum, Brassica napus, Brassica oleracea, Brassica rapa, Brassica juncacea, Brassica nigra, Glycine max, Gossypium sp., Sorghum bicolor, Triticale, Saccharum officinarium, Setaria italica, Oryza sativa, Oryza minuta, Oryza australiensis, Oryza alta, Brachypodium distachyon, Hordeum marinum, Aegilops tauschii, Daucus glochidiatus, Daucus pusillus, Daucus muricatus, Daucus carota, Eucalyptus grandis, Erythranthe guttata, Genlisea aurea, Musa sp., Avena sp., Nicotiana sylvestris, Nicotiana tabacum, Nicotiana tomentosiformis, Solanum lycopersicum, Coffea canephora, Vitis vinifera, Cucumis sativus, Morus notabilis, Crucihimalaya himalaica, Crucihimalaya wallichii, Cardamine flexuosa, Lepidium virginicum, Capsella bursa-pastoris, Olmarabidopsis pumila, Arabis hirsuta, Raphanus sativus, Eruca vesicaria sativa, Citrus sinensis, Jatropha curcas, Populus trichocarpa, Beta vulgaris and Manihot esculenta.
[9] A population of a polyploid plant as defined in any one of [1] to [8].

Furthermore, the present invention provides populations of polyploid plants as characterized above, wherein a population comprises 7, 10, 20, 50, 100, 500, 1000, 2000, 5000, 10000 or even more of said polyploid plants and wherein the proportion of non-polyparental plants is 75% at the most, but preferably less than 50%, more preferably less than 25%, even more preferably less than 10%, and most preferably less than 1%.
[10] Organ, part, tissue, cell, seed or offspring of the plant of any one of [1] to [8].

In a further embodiment, the present invention also relates to organs, parts, tissues, cells, seed or offspring of the polyploid plant of the present invention.
[11] A method for generating a polyploid plant of any one of [1] to [8] comprising
   (a) providing at least three parental plants, wherein at least one first and one second male parent plant comprises a component of a selectable marker system;
   (b) crossing the at least three parental plants;
   (c) selecting the offspring obtained by step (b) for the presence of the marker; and optionally
   (d) generating marker-free plants via
      (i) crossing or selfing at least two of the offspring of step (c); and
      (ii) selecting the offspring of step (d)(i) for the absence of the marker.

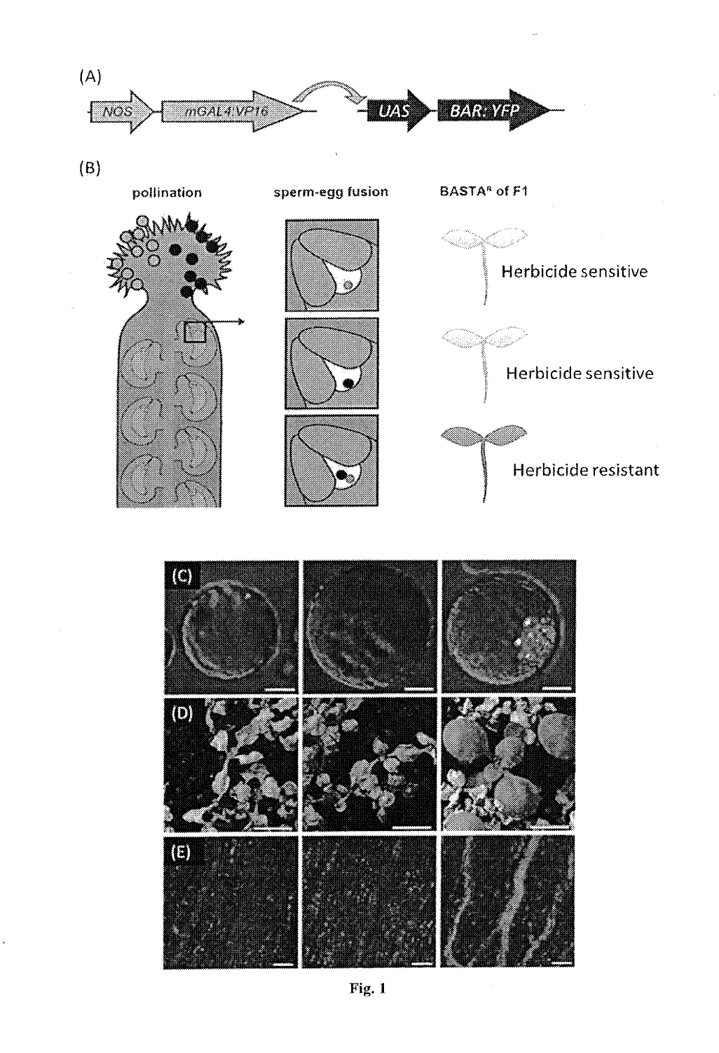

The method of the present invention, i.e. HIPOD method is illustrated in FIG. 1 and comprises as a first step the generation of at least two transgenic male parent plants or plant lines each of which comprises a component which together during sperm-egg fusion gives rise to a selectable marker expressed in the offspring. The components of the selectable marker system preferably employed in accordance with the present invention are derived from the so called yeast two-hybrid system initially developed for studying protein-protein and protein-nucleic acid interactions, which meanwhile also has been established as a means for controlling gene expression in plants; see, e.g., the expression cassettes described in European patent application EP 0 589 841 A2. Furthermore, the two-hybrid system has been further developed into a tri-/three-hybrid system; see, e.g., Putz et al., Nucleic Acids Research 24 (1996), 4838-4840 and Cottier et al., Front Plant Sci. 2 (2011): 101. The three-hybrid system may be adapted similar as the two-hybrid system in accordance with the method of the present invention, thus providing three components allowing the cross of three male parent lines, each of which carries one of the three components, with a female plant resulting in a tetra-parental and for example tetraploid or octaploid plant. Furthermore, the components of the selectable marker system employed in accordance with the present invention can also be derived from the transcription activation system for regulated gene expression in plants, which was initially described by Moore et al., Proc. Natl. Acad. Sci. USA 95 (1998), 376-381 and has been adapted and improved over the years. Based on this, the pOp6/LhGR and pOp/LhG4 systems for regulated gene expression have been established and are known in the art; see, e.g. Craft et al., Plant J. 41 (2005), 899-918 and Samalova et al., Plant J. 41 (2005), 919-935. Alternatively, the components of the selectable marker system may consist of two different selectable markers, for example a herbicide and antibiotic conferring resistance gene which when combined through fusion of the sperm cells and the egg cell gives rise to plants resistant to the at least two selectable marker.

Thus, the method of the present invention may encompass the following steps:
(a1) transforming a first parent plant cell with the first component, e.g. an expression cassette, said cassette comprising a nucleotide sequence encoding a 5′-regulatory region e.g. a promoter active in plant cells operably linked to a nucleotide sequence which encodes a transactivator polypeptide; and regenerating a transformed plant, parent 1, from said first transformed plant cell;
(a2) transforming a second parent plant cell with the second component, e.g. expression cassette comprising a target nucleotide sequence which is capable of being activated by said transactivator polypeptide operably linked to a nucleotide sequence which encodes a selectable marker, for example which provides antibiotic, herbicide and/or antimetabolite resistance when expressed; regenerating a transformed plant, parent 2, from said second transformed plant cell; and
(b) crossing said parent 1 and parent 2 as the male parent plants with a female plant to obtain polyploid, triparental offspring.

As mentioned, in principle the means and methods described in European patent application EP 0 589 841 A2, for example the expression cassettes, may be used in the method of the present invention except that instead of an anther-specific 5′-regulatory region a constitutive promoter such as the 35S or NOS promoter employed in the Examples may be used and instead of a nucleotide sequence which encodes anti-sense RNA or a polypeptide which disrupts the formation of viable pollen when expressed, a nucleotide sequence is used encoding a product that provides for a selectable phenotype such as the BAR gene or a fluorescent protein. Thus, the components necessary and sufficient to arrive at the marker system for use in accordance with the present invention are well known in the art. Furthermore, as mentioned, more than two male parent plants may be used for generating multi-parental plants, for example using a three-hybrid component system which may comprise the following steps:
(a1) transforming a first parent plant cell with the first component, e.g. an expression cassette, said cassette comprising a nucleotide sequence encoding a 5′-regulatory region e.g. a promoter active in plant cells operably linked to a nucleotide sequence which encodes a first part of a transactivator polypeptide comprising a dimerization domain; and regenerating a transformed plant, parent 1, from said first transformed plant cell;
(a2) transforming a second parent plant cell with the second component, e.g. an expression cassette, said cassette comprising a nucleotide sequence encoding a 5′-regulatory region e.g. a promoter active in plant cells operably linked to a nucleotide sequence which encodes a second part of a transactivator polypeptide comprising a dimerization domain effective to dimerize with the dimerization domain of the first part of a transactivator polypeptide of (a1); and regenerating a transformed plant, parent 2, from said second transformed plant cell;
(a3) transforming a third parent plant cell with the third component, e.g. an expression cassette comprising a target nucleotide sequence which is capable of being activated by the transactivator polypeptide dimer of (a1) and (a2) operably linked to a nucleotide sequence which encodes a selectable marker, for example which provides antibiotic, herbicide and/or antimetabolite resistance when expressed; regenerating a transformed plant, parent 3, from said third transformed plant cell; and
(b) crossing said parent 1, parent 2 and parent 3 as the male parent plants with a female plant to obtain polyploid, tetra-parental offspring.

Thus, in principle steps (a1) and (a2) may be extended to (a2+n) to obtain polyploid, (2+n)+1-parental offspring by designing the marker system such that (2+n) components must act in an concerted manner in order to give rise to the selectable marker when combined in one single plant.

In an alternative embodiment, the method of the present invention comprises as a first step the generation of at least two transgenic male parent plants or plant lines each of which comprises a different selectable marker which together during sperm-egg fusion gives rise to plants resistant to the at least two selectable markers.

Thus, the method of the present invention may encompass the following steps:
(a1) transforming a first parent plant cell with the first component, e.g. an expression cassette, said cassette comprising a nucleotide sequence encoding a 5′-regulatory region e.g. a promoter active in plant cells operably linked to a nucleotide sequence which encodes a first selectable marker; and regenerating a transformed plant, parent 1, from said first transformed plant cell;
(a2) transforming a second parent plant cell with the second component, e.g. an expression cassette, said cassette comprising a nucleotide sequence encoding a 5′-regulatory region e.g. a promoter active in plant cells operably linked to a nucleotide sequence which encodes a second selectable marker; and regenerating a transformed plant, parent 2, from said first transformed plant cell; and
(b) crossing said parent 1 and parent 2 as the male parent plants with a female plant to obtain polyploid, triparental offspring.

Furthermore, as mentioned, more than two male parent plants may be used for generating multi-parental plants being resistant to multiple selectable markers.

Advantageously, the F1 offspring of the method of the present invention is heterozygous for the selectable marker gene but “homozygous” as regards its polyploidy, e.g. triparental. Typically, triploid hybrids with 3 sets of chromosomes (ABD), and plants with an uneven set of chromosomes in general have a reduced fertility and may be sterile. However, as described in Example 4 and illustrated in FIG. 3H triparental plants produced in accordance with the present invention show fertile ovules and thus may be amenable to propagation by selfing. Furthermore, fertile F1 progeny may be arrived at due to the generation of unreduced gametes or somatic doubling of the set of chromosomes. For example, a sterile hybrid seedling can be treated with a G2/M cell cycle inhibitor, e.g. a microtubule polymerization inhibitor such as colchicine, nocodazole, 5 oryzaline, trifluraline and vinblastine sulphate to produce a plant with twice as many chromosomes in order to restore fertility. The use of colchicine to produce a plant with twice as many chromosomes, for example from sterile triploid rye/wheat hybrid with 3 sets of chromosomes to produce a fertile hexaploid (6n) version of triticale is well known in the art and meanwhile applied for other plants as well; see, e.g., international applications WO 2013/011507 A1 for maize, WO 2009/095266 A1 for eggplant, WO 2012/145248 A1 for Miscanthus x giganteus and Faleiro et al., Plant Cell, Tissue and Organ Culture (PCTOC) 124 (2016), 57-67.

The selfing of the hexaploid version of the triparental plant would then result in a typical F2 segregation of the offspring including plants which are triparental but free of the components of the marker gene system, i.e. free of the (different) marker(s) or parts thereof and thus non-transgenic. Accordingly, in an additional step the method of the present invention comprises the selfing of the F1 generation inter alia resulting in marker gene-free F2 plants devoid of any DNA which is foreign to the plant. Alternatively, meanwhile techniques exist to generate marker-free transformed/transgenic plants, for example the CRISPR-Cas system (see, e.g., Lowder et al., Plant Physiology 169 (2015), 971-985) and the cre/lox site-specific recombination (see, e.g., Wang et al., Transgenic Res. 14 (2005), 605-614) which though less preferred may be used for generating marker-free plants.

Thus, the present invention generally relates to a method for the generation of polyparental polyploid plants comprising (a) providing more than two parental plants, wherein (i) each of the male parent plants comprises a part of a components of a selectable marker system, or (ii) each of the male parent plants comprises a different selectable marker; (b) crossing the more than two parental plants; (c) selecting the offspring obtained by step (b) for the presence of the marker(s); and optionally (d) generating marker-free plants via (i) crossing or selfing at least two of the offspring of step (c); and (ii) selecting the offspring of step (d)(i) for the absence of the marker(s).

Means and methods for generating transgenic plants including the transformation of dicotyledonous and monocotyledonous plants are known in the art, see, e.g., Plant Cell and Tissue Culture—A Tool in Biotechnology: Basics and Application, Springer 2009 ISBN 978-3-540-93883-5. Efficient promoter cassettes for enhanced expression of foreign genes in dicotyledonous and monocotyledonous plants are well known in the art; see, e.g., Mitsuhara et al., Plant Cell Physiol. 37 (1996), 49-59 and Potenza et al., In Vitro Cellular & Developmental Biology—Plant 40 (2004), 1-22, the latter for review. Likewise, engineering herbicide resistance in plants by expression of a detoxifying enzyme is well known since 1980ths; see, e.g., De Block et al., EMBO J. 6 (1987), 2513-2518 and Miki et al., J. Biotechnol. 107 (2004), 193-232, the latter for review. Specific examples for the construction of the plant vectors and the generation of the transgenic plants of the present invention are described in Examples 1, 2 and 3 and can be adapted according to the selectable markers of interest.

Thus, in one embodiment, the method of the present invention generally relates to a method comprising the crossing of one female parent plant with more than one male parent plant, wherein the male parent plants each comprise a part of a selectable marker system which when present in one single plant allows for selection of said plant.

After crossing of the transgenic male parent plants comprising the individual part of the selectable marker system with a female parent plant, selection of the seedlings is performed. Seedlings resulting from monospermy, i.e. from crossing of the female parent plant with only one male parent plant are rendered sensitive for the selectable marker. In contrast, combinations of both constructs, which can only result from polyspermy will give rise to plants resistant to the selectable marker or which show a selectable phenotype.

In an alternative embodiment, the method of the present invention comprises the crossing of one female parent plant with more than one male parent plant, wherein the male parent plants each comprise a different selectable marker which when present in one single plant allow for selection of said plant for the presence of these markers.

After crossing of the transgenic male parent plants comprising the different selectable markers with a female parent plant, selection of the seedlings is performed. Seedlings resulting from monospermy, i.e. from crossing of the female parent plant with only one male parent plant are are only resistant to the one selectable marker comprised in one male parent plant. In contrast, combinations of both constructs, which can only result from polyspermy will give rise to plants resistant to the different selectable markers or which show a selectable phenotype. Preferably, a first selectable marker is used conferring herbicide resistance, preferably resistance to an herbicide that inhibits the glutamine synthase, such as the glufosinate-based herbicide BASTA (Biolophos) and a second selectable marker is used conferring antibiotic resistance, preferably resistance to hygromycin.

Preferably, said methods allow the crossing of one female parent plant with two, three, four, five, six, seven, eight, nine or ten male parents plants, wherein the crossing can occur between parent plants with different ploidy, i.e. they can be haploid, diploid, triploid, tetraploid, pentaploid, hexaploid, heptaploid, octaploid, nonaploid, decaploid, undecaploid or dodecaploid. Thus, the selectable marker system consists of two, three, four, five, six, seven, eight, nine or ten parts or of two, three, four, five, six, seven, eight, nine or ten different selectable markers, which when combined in one single plant allows for selection of said plant.

In a preferred embodiment, the method of the present invention comprises the crossing of one diploid or tetraploid female parent plant with two diploid or tetraploid male parent plants, wherein the two male parent plants comprise each a part of the selectable marker system; thus said maker system consists of two parts either of one selectable marker or alternatively of two different selectable marker.

In one embodiment, the selectable marker system of the present invention comprises a first part comprising a driver expressing a heterologous transcription factor under the control of a ubiquitous promoter and a second part comprising the selectable marker gene(s) under the control of a corresponding responsive promoter. More particular, when combined in one plant, preferably in close proximity, i.e. in the same part of the plant, the individual parts of the selection system resemble to a functional selectable marker and/or to a functional expression unit expressing the gene product which serves as selectable marker comprising the selectable marker gene operably linked to a regulatory sequence, preferably to a promotor and/or a heterologous transcription factor.

Since transgenic plants are obtained by the method of the present invention (steps (a) to (c)), which are still under debate in the public and have poor acceptance in particular with respect to food, said method provides the possibility to generate marker-free, non-transgenic plants (steps (d) and (e)) via crossing or selfing of the F1 generation resulting in marker gene-free plants that are devoid of any DNA which is foreign to the plant.
[12] The method of [11], wherein the selectable marker confers herbicide, antibiotic and/or antimetabolite resistance and/or a detectable, preferable visible output.

In one embodiment, the selectable marker of the present invention confers herbicide resistance. Preferably, the herbicide can be selected from but is not limited to a herbicide that inhibits acetyl coenzyme A carboxylase, such as aryloxyphenoxypropionate (FOPs)-, cyclohexanedione (DIMs)-, and phenylpyrazolin (DENs)-based herbicides, a herbicide that inhibits acetolactate synthase including sulfonylureas (such as flazasulfuron and metsulfuron-methyl), imidazolinones, triazolopyrimidines, pyrimidinyl oxybenzoates, and sulfonylamino carbonyl triazolinones, a herbicide that inhibits the 5-enolpyruvylshikimate-3-phosphate synthase EPSPS, such as glyophysate-based herbicides (Roundup), a herbicide that inhibits the glutamine synthase, such as the glufosinate-based herbicide BASTA (Biolophos), a synthetic auxin herbicide, such as 2,4-D and Dicamba, a herbicide that inhibits the photosystems, including the triazine herbicides, such as atrazine, the urea derivatives, such as diuron, the bipyridinium herbicides, such as diquat and paraquat, the diphenyl ether herbicides, such as nitrofen, nitrofluorfen, acifluorfen, and oxyfluorfen, and the nitrile-herbicides, such as bromoxynil-based herbicides, and a herbicide that inhibits the phytoen-desaturase, including norflurazon-based, fluridon-based and difunon-based herbicides. Preferably, the herbicide is a glufosinate-based herbicide such as the non-selective herbicide (“total weedkiller”) BASTA. The antibiotic can be selected, but is not limited to kanamycin, neomycin, geneticin, paromomycin, hygromycin, gentamicin, spectinomycin, streptomycin or tobramycin.

In a further embodiment, the selectable marker of the present invention confers a detectable, preferably visible output such as fluorescence when the parts of the selectable marker system are brought into close proximity, i.e. are present in one plant, preferably in one part of the plant.

In a further embodiment, the selectable marker system of the present invention comprises the selectable marker conferring herbicide, antibiotic and/or antimetabolite resistance as well as a selectable marker conferring the visible detectable output, wherein the first one is preferably used for selection of the plants and the second one is preferably used as reporter for visualization. In a preferred embodiment, the selectable marker confers herbicide resistance and a reporter gene is used for visualization.

Thus, after crossing of the transgenic male parent plants comprising the individual part of the selectable marker system, i.e. either of one selectable marker or alternatively of two different selectable marker with a female parent plant, selection of the seedlings is performed with an herbicide. Seedlings resulting from monospermy, i.e. from crossing of the female parent plant with only one male parent plant are rendered sensitive for the herbicide and die upon herbicide treatment and/or remain colorless. In contrast, combinations of both constructs, which can only result from polyspermy will give rise to herbicide resistant and/or fluorescent progeny.

Of course, next to the selectable markers conferring herbicide resistance, further selectable markers for plants commonly known in the art can be used as well, e.g. selectable markers conferring resistance to antibiotics or antimetabolites and further markers conferring a visible output can be used, for example the lacZ system.
[13] The method of [11] or [12], wherein the at least one first and one second male parent plant are homozygous for the components.

Since plants are eukaryotic organisms, they possess two or more copies of its genetic information per cell. Usually, every gene is represented by two alleles, which are identical in the homozygous state and are different in the heterozygous state, respectively. In a further embodiment, the male parent plants are homozygous for the individual parts of the selectable marker system. In particular, the first male parent plant (first pollen donor) is homozygous for a construct comprising a driver expressing a heterologous transcription factor under the control of an ubiquitous promoter and wherein the second pollen donor is homozygous for a construct comprising a herbicide resistance conferring gene, preferably the BAR gene or the pat gene under the control of a corresponding responsive promoter, optionally wherein the herbicide resistance conferring gene is linked to a fluorescence conferring gene, preferably the YFP gene. Alternatively, the male parent plants are homozygous for the different selectable marker.
[14] The method of [12] or [13], wherein the selectable marker confers herbicide, antibiotic and/or antimetabolite resistance and/or a visible detectable output, preferably wherein the selectable marker is selected from a herbicide resistance gene selected from the group consisting of the bar gene and the pat gene, and/or an antibiotic resistance gene selected from the group consisting of the nptII gene, the hpt gene, the acc3 gene, the aadA gene, and/or a gene conferring antimetabolite resistance such as the dhfr gene, and/or a bioreporter gene selected from the group consisting of a GFP gene, a YFP gene, a CFP gene, a BFP gene, a Venus gene, a dsRED gene, a hcRED gene, a mCherry gene, a mPlum gene, a mOrange gene, a zoanFP gene, a Cerulan gene, a luc gene, a cat gene, lacZ gene and uidA gene as well as modified and/or enhanced versions thereof.

The selectable marker system of the present invention is adapted to the mGAL4-VP16/UAS-based system well known in the art; see e.g. European patent application EP 0 589 841 A2. Consequently, the heterologous transcription factor of the construct of the first male parent plant (first pollen donor) is mGAL4-VP16 and the promoter is the NOS promoter or the RPS5a promoter and wherein the responsive promoter of the construct of the second male parent plant (second pollen donor) is the UAS promoter. Thus, both selectable markers, the one conferring herbicide resistance and the other conferring fluorescence are under control of the same regulatory elements. Of course, equivalent marker systems may be used as well for example those based on the first and second expression cassettes described in European patent application EP 0 589 841 A2. As mentioned, preferably the selectable marker gene confers herbicide resistance, most preferably the selectable marker system of the present invention comprises the BAR gene conferring resistance to the glufosinate-based herbicide BASTA.

The fluorescence conferring selectable marker, also referred to as reporter, can be of any kind as long as it confers an observable or measurable phenotype. According to the present invention, the green fluorescent protein (GFP) from the jellyfish Aequorea victoria (described in international applications WO95/07463, WO96/27675 and WO95/121191) and its derivates “Blue GFP” (Heim et al, Curr. Biol. 6 (1996), 178-182), Redshift GFP” (Muldoon et al., Biotechniques (1997), 162-167) as well as enhanced “EGFP” variants can be used. Further embodiments are the yellow, cyan and blue fluorescent proteins (YFP, CFP and BFP, respectively) as well as its enhanced variants EYFP, ECFP and EBFP, the red fluorescent proteins (DsRed, HcRed, mCherry, dtTOMATO) and further fluorescent proteins such as the Venus protein, mPlum, mOrange, zoanFP and Cerulan. Further fluorescent proteins are known to the person skilled in the art and can be used according to the invention. The detection of fluorescent proteins takes place through per se known fluorescence detection methods. A summary of fluorescent proteins used in transgenic plants as well as methods for detection is for example provided in “Fluorescent Proteins in Transgenic Plants by Reginald J. Millwood, Hong S. Moon, and C. Neal Stewart Jr.”. In a preferred embodiment, the YFP gene is used as selectable marker/reporter.
[15] A polyploid plant obtainable by the method of any one of [11] to [14] or part, tissue, cell, seed or offspring of the plant.

Naturally, the present invention also extends to the plants obtained and obtainable, respectively, by the method of the present invention or any of the individual steps (a1+n) as well to parts, tissue, cells, seed and offspring as well as progeny of those plants, including the following:
[16] Use of a first transgenic plant in the method of the present invention, wherein the transgenic plant comprises either a stably integrated expression cassette wherein said expression cassette comprises a nucleotide sequence encoding a promoter, preferably constitutive promoter which is operably linked to a nucleotide sequence encoding a transactivator (or part thereof) capable of regulating a target nucleotide sequence or a stably integrated first expression cassette comprising a nucleotide sequence encoding a promoter, preferably constitutive promoter which is operably linked to a nucleotide sequence encoding a first selectable marker, preferably which provides antibiotic, herbicide and/or antimetabolite resistance and/or reporter gene expression when expressed.
[17] Use of a second transgenic plant in the method of the present invention, wherein the second transgenic plant comprises either a stably integrated expression cassette wherein said expression cassette comprises a target nucleic acid sequence, which is capable of being activated by a transactivator, operably linked to a nucleotide sequence which encodes a selectable marker, preferably which provides antibiotic, herbicide and/or antimetabolite resistance and/or reporter gene expression when expressed or a stably integrated second expression cassette comprising a nucleotide sequence encoding a promoter, preferably constitutive promoter which is operably linked to a nucleotide sequence encoding a second selectable marker, preferably which provides antibiotic, herbicide and/or antimetabolite resistance and/or reporter gene expression when expressed.
[18] A population of plants comprising the at least three parental plants as defined in any one of [11] to [14], preferably the transgenic plants of [16] and [17] and a third, preferably non-transgenic plant, preferably under conditions allowing pollination of said third plant as the female parent plant by pollen of said first and second transgenic plant as the male parent plants.
[19] A transgenic tri- or multiparental plant and the progeny thereof, which comprises either a stably integrated first expression cassette comprising a nucleotide sequence encoding a promoter, preferably constitutive promoter which is operably linked to a nucleotide sequence encoding a transactivator and a second expression cassette comprising a target nucleotide sequence, which is activated by said transactivator polypeptide, operably linked to a nucleotide sequence which encodes a selectable marker, or a stably integrated first expression cassette comprising a nucleotide sequence encoding a promoter, preferably constitutive promoter which is operably linked to a nucleotide sequence encoding a first selectable marker and a second expression cassette comprising a nucleotide sequence encoding a promoter, preferably constitutive promoter which is operably linked to a nucleotide sequence encoding a second selectable marker, preferably wherein the selectable marker provides antibiotic, herbicide and/or antimetabolite resistance when expressed.

These and other embodiments are disclosed and encompassed by the description and Examples of the present invention. Further literature concerning any one of the materials, methods, uses, and compounds to be employed in accordance with the present invention may be retrieved from public libraries and databases, using for example electronic devices. For example the public database “Medline” may be utilized, which is hosted by the National Center for Biotechnology Information (NCBI) and/or the National Library of Medicine at the National Institutes of Health (NLM.NIH). Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2. Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention. A more complete understanding can be obtained by reference to the following specific examples which are provided herein for purposes of illustration only and are not intended to limit the scope of the invention.

### EXAMPLES

In the following, specific materials and methods used in context with the present invention are listed.

### Plant Materials and Growth Conditions

Wild-type Arabidopsis strains used are Ler, Col-0, and C24. Plants were germinated on soil in a Conviron MTPS growth chamber under long-day conditions (16 hr light/8 hr dark) at 23° C. Upon bolting, plants were transferred to 18° C. both at otherwise constant growth conditions. Plant selection was either performed on soil by spraying the herbicide BASTA, or seeds were surface sterilized with ethanol and plated on MS medium containing hygromycin.

### Statistical Analysis

No statistical methods were used to predetermine sample size. The experiments were not randomized, and investigators were not blinded during experiments and analysis. Statistics were performed using two tailed t-test for unequal variances: * p<0.05; ** p<0.01; ***p<0.001.

### Example 1: Cloning of the Vector Constructs Used for Establishment of a High-Throughput Polypaternal Breeding Design (HIPOD) Method

The HIPOD constructs have been generated via adapting the mGAL4-VP16/UAS-based system, comprising a driver expressing the heterologous transcription factor mGAL4-VP6 under the control of the ubiquitous RPS5a promoter described by Haselhoff, Methods Cell Biol. 58 (1999), 139-151; see FIG. 1 (A). The HIPOD constructs were assembled on the basis of a binary pGreenII 0176 (Genbank: EU048866 http://www.ncbi.nlm.nih.gov/nuccore/EU048866) containing a hygromycin and kanamycin resistance available at http://www.pgreen.ac.uk (Hellens et al., Plant Molecular Biology 42 (2000), 819-832; Hellens, pGreen II 2007). The multiple cloning site was altered to include additional AscI and PacI restriction sites cloned in front of SpeI, NotI and SacI into the MCS. The respective sequences for the UAS/GAL4: VP16 transactivation system were amplified via PCR (Haseloff, Methods Cell Biol. 58 (1999), 139-151; Sadowski et al., Nature 335 (1988), 563-564). The UAS sequence was amplified using TN13s (5′-ATGGCGCGCCGCATGCCTGCAGGTCGGA-3′) (SEQ ID NO: 1) and TN13 as (5′-ATTTAATTAACGGGGATCCGGTTCTCTC-3′) (SEQ ID NO: 2). Subsequently YFP (Boisnard-Lorig et al., The Plant Cell 13 (2001), 495-509) was cloned into the aforementioned pGreenII 0176 vector using NotI/SacI restriction sites. The BAR gene (Thompson et al., The EMBO Journal 6, (1987), 2519-2523 was amplified via PCR from pGreenII 0229 (GenBank: EU048867.1/http://www.ncbi.nlm.nih.gov/nuccore/EU048867) vector using primer TN14s (5′-ATTTAATTAAATGAGCCCAGAACGACGCCC-3′) (SEQ ID NO: 3) and TN14as (5′-ATGCGGCCGCGATTTCGGTGACGGGCAGGAC-3′) (SEQ ID NO: 4) and integrated into the vector using PacI/NotI. In a final step UAS was fused N-terminally using AscI/PacI. GAL4:VP16 was amplified by PCR using TN12s (5′-ATTTAATTAAATGAAGCTCCTGTCCTCCATCGA-3′) (SEQ ID NO: 5) and TNI2as (5′-ATGCGGCCGCCTACCCACCGTACTCGTCAATTC-3′) (SEQ ID NO: 6). GAL4:VP16 was inserted into the altered pGreenII 0176 backbone using PacI/NotI restriction sites. PRPS5a was amplified from the Arabidopsis genome using TSI5s (5′-AGGCGCGCCGGGCCATAATCGTGAGTAGAT-3′) (SEQ ID NO: 7) and TS15as (5′-ACGATCGCGGCTGTGGTGAGAGAAACA-3′) (SEQ ID NO: 8) and cloned into © pGemT-easy vector system (Promega) (Weijers et al., Nature 414 (2001), 709-710; Weijers et al., Development 128, (2001), 4289-4299). Subsequently pRPS5a was digested using AscI/PvuI and N-terminally fused to the aforementioned pGreenII backbone containing mGAL4:VP16 digested with AscI/PacI. The nucleotide sequence of the gene conferring resistance to BASTA is shown in SEQ ID NO: 9. The nucleotide sequence of the promoters UAS and RPS5a are shown in SEQ ID NO: 10 and 11, respectively. The nucleotide sequence of the YFP gene is shown in SEQ ID NO: 12 and the sequence of the GAL4:VP16 construct is shown in SEQ ID NO: 13.

### Example 2: Establishing the HIPOD Method

First, the system was tested in a transient expression assay using Arabidopsis protoplasts. Transformation with only one of the constructs yielded YFP-negative protoplasts. By contrast, protoplasts that have been co-transfected with both HIPOD constructs exhibit YFP fluorescence; see FIG. 1 (C). In the next step, distinct Arabidopsis thaliana (Ler) plants were transformed with the individual binary vector constructs (pRPS5a::mGAL4-VP16 and UAS::BAR_YFP) by Agrobacterium tumefaciens assisted floral dipping. Plants homozygous for the pRPS5a::mGAL4-VP16 construct constitute pollen donor 1 (PD1) and plants homozygous for the responder construct UAS::BAR_YFP constitute pollen donor 2 (PD2). While the propagation of either PD1 or PD2 resulted in herbicide sensitive plants, reciprocal crosses between PD1 and PD2 yielded herbicide resistant and YFP-positive progeny; see FIGS. 1 (D) and (E).

### Example 3: Implementation of HIPOD for the Generation of Polyploid, Triparental Plants

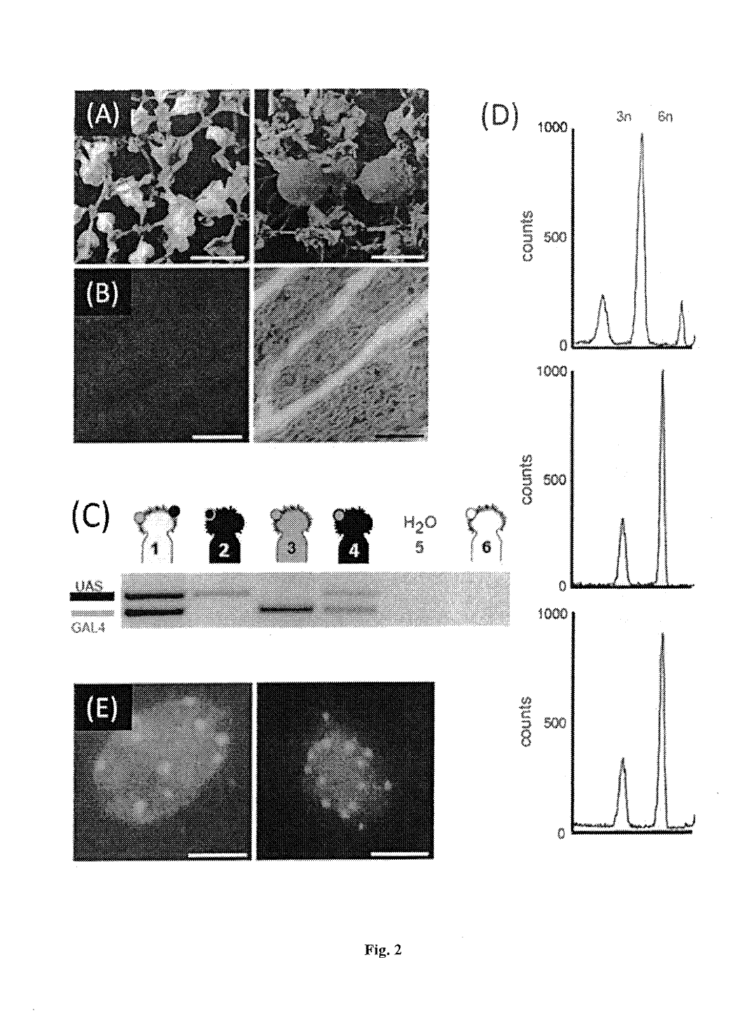

For the generation of triparental plants, PD1 (homozygous for the pRPS5a::mGAL4-VP6 construct) and PD2 (homozygous for the responder construct UAS::BAR_YFP) were provided as distinct pollen donors onto wild-type flowers. Therefor pollen grains from PD1 and PD2 were independently collected using a vacuum cleaner based collection device adopted from Jose Feijo lab (Johnson-Brousseau and McCormick, Plant. J. 39 (2004), 761-775) and the 3-8 oldest closed flower buds were emasculated and pollinated after two to three days using a brush. The procedure was repeated 4 times and in total 2575 double pollinations were performed yielding 120644 seeds. The seeds can be counted using open source imaging processing software, such as ImageJ (http://imagej.net/; e.g. Schneider et al., Nature methods 9 (2012), 671-675. Following herbicide treatment of the F1, 7 herbicide (BASTA) resistant seedlings have been detected; see FIG. 2 (A). Furthermore, dissected cotyledons or sepals from opened flowers were transferred to 10% glycerol and analyzed with Leica DMI6000b fluorescence microscope using standard protocols. Upon this inspection, YFP fluorescence has been detected (FIG. 2 (B)), which is specifically associated with the BAR gene used in the HIPOD method.

To determine whether these plants were indeed of triparental origin, the seven seedlings were subjected to genotyping by multiplex PCR using primers 5′-TATAGGGCGAATTGGGTACC-3′ (SEQ ID NO: 14) and 5′-GGAACTGGCATGACGTGGGTTT-3′ (SEQ ID NO: 15) that target the PD2 construct (UAS::BAR_YFP) as well as primers 5′-TCGTTTTCTCTGCCGTCTCTCT-3′ (SEQ ID NO: 16) and 5′-CCCTTGTTGCTGCTCTCCTC-3′ (SEQ ID NO: 17) that target the PD1 construct (pRPS5a:: mGAL4-VP16). Importantly, all plants segregated both HIPOD constructs (FIG. 2 (C)), indicating that the genetic material of two different fathers had been transmitted to a single egg cell.

The introgression of the two paternal copies implies that the resulting plants are triploid. In order to determine the ploidy of the seedlings, flow-cytometry has been performed. For this, one or two leaves were harvested and chopped using a razor blade in a petri dish with nuclei extraction buffer (Partec CyStain®). Afterwards, staining reagent (Partec CyStain UV Precise-Kit®) was added and incubated at RT for 1 min. The liquid was passed through a 50 μM nylon mesh and analyzed using Partec CyFlow® ploidy analyzer. Notably, all seven plants exhibited a profile characteristic to triploid plants; see FIG. 2 (D). To confirm this result, a complementary approach was performed, in which the chromosome number of the herbicide-resistant plants was determined making use of chromosomal spreads. Chromosome spreads were carried out by analyzing flower buds taken from representative plants of each genotype according to published protocol (Heslop-Harrison in Arabidopsis: A practical approach—The Practical Approach Series (ed. Zoe A. Wilson), Chapter 5, 105-123, Oxford University Press). Arabidopsis thaliana contains five different chromosomes and in the diploid Landsberg erecta accession used in this study they exist in two copies. Notably, in nuclei of the herbicide-resistant offspring recovered from HIPOD, 15 instead of 10 chromosomes have been detected, confirming the triploid nature of these plants; see FIG. 2 (E).

### Example 4: Characterization of Growth and Viability of the Triparental Plants (Plant Phenotyping)

Growth height, sepal length, petal length and cell size were measured from digital images. Dissected sepals and petals were mounted in 70% alcohol to measure proximodistal dimensions. For cell size measurements petals were cleared in chloral hydrate:glycerine:ddH₂O (8:2:1) and average cell sizes were calculated at the adaxial side, from the number of cells per unit area of digital micrographs as described in Disch et al., Curr. Biol. 16 (2006), 272-279. All size measurements were calculated using ImageJ software. Fertility assay was carried out as described in Volz et al., Dev. Cell 25 (2013), 310-316. The total number of seeds produced per wild-type plant was assessed by collecting all mature siliques during the plants life-time. Digital images of the respective seeds were processed the above mentioned software tool.

In order to discriminate attributes caused by the introgression of additional paternal copies and potential negative effects specifically associated with the unique polyspermic origin of these plants, biparental triploid plants have been included, which have been recovered from an interploidy cross (2n×4n). In comparison to diploid plants, overall plant size of triparental plants was increased 1.5 fold and the plants gave rise to bigger inflorescences and flowers. The size increase was comparable to that of biparental triploid plants; FIG. 3 (A)-(D). In addition, it was found that triparental plants produced significantly larger organs when compared to diploid plants but exhibited similar organ size when compared to biparental triploids. Petal and sepal size were increased by 20.7% and 17.0% respectively; FIGS. 3 (E) and (F). To determine whether organ hypertrophy was caused by cell proliferation or expansion, the size of petal epidermis cells has been determined. Cell size in both triparental and biparental triploids was substantially increased; see FIGS. 3 (G) and (J). An inherent consequence of meiosis in triploid plants is aneuploidy, i.e. an imbalance in chromosome number. In fact, it has been observed that fertility in triparental plants and biparental triploids is substantially reduced accompanied by the segregation of shriveled and malformed ovules and seeds; see FIGS. 3 (H), (I) and (K). The data suggest that the differences between biparental diploid and polyspermy-induced triploid plants described in this study are mainly due to the inheritance of an extra paternal genomic copy, and that the polyspermic origin per se does not impose gross negative impacts on development and growth.

### Example 5: Calculation of the Polyspermy Frequency in the HIPOD Method

As an approximation, the polyspermy rate was calculated on the basis of total seed counts (120644) and the number of recovered triparental seedlings (7), as evidenced by BASTA resistance and positive identification of both paternal HIPOD constructs. In addition, it was taken into account that via using the HIPOD method, only ⅓ of all polyspermy events can be selected as supernumerary sperm contributions involving sperm from single father escape detection: Frequency=(3×# triparental plants/# seeds)×100. Thereby, a polyspermy frequency of 0.017% has been yielded. When extrapolating this polyspermy frequency to the approximately 45000 seeds generated on average by Arabidopsis under the growth conditions used in this study, the data imply that every plant can give rise to about 7 polyspermy-induced polyploid plants.

### Example 6: Instant Hybridization of Three Different Germplasms

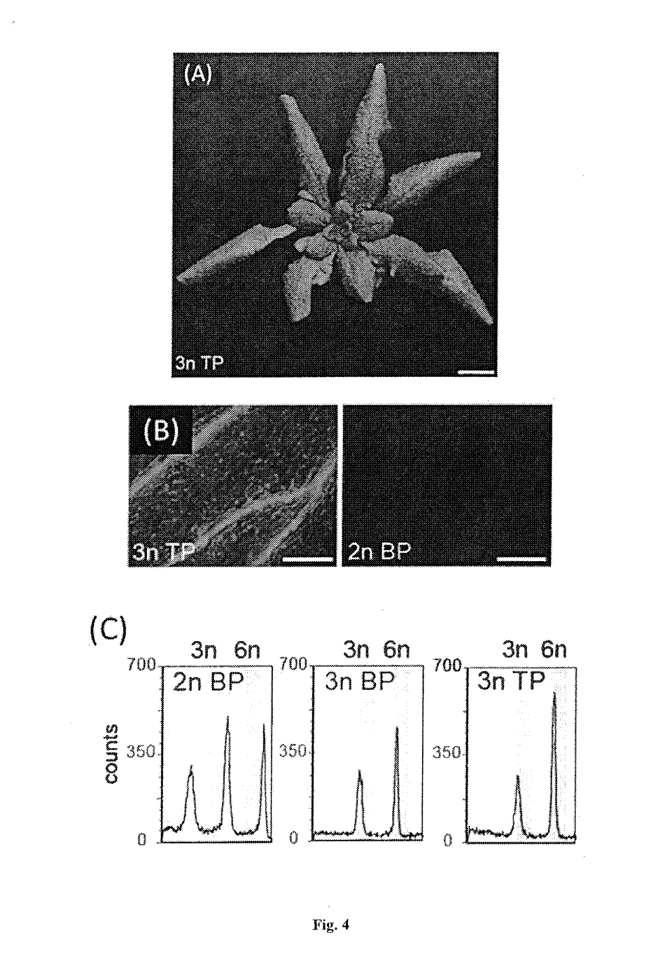

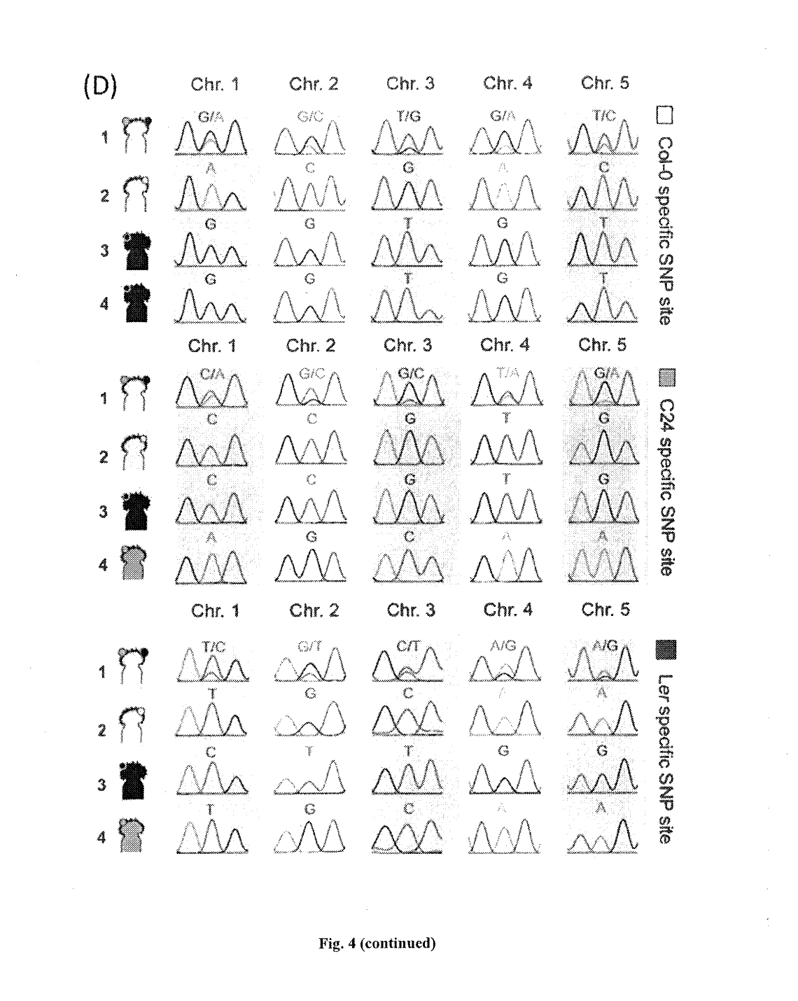

Three genetically distinct Arabidopsis accessions have been combined in a three parent cross. For this three accession HIPOD cross more than 200 double pollinations were performed in two independent experiments, yielding 9493 seeds. In order to facilitate the detection of triparental offspring, the UAS HIPOD responder construct (see Example 1) has been introduced into the Arabidopsis Ler accession and the GAL4 HIPOD driver construct (see Example 1) into the C24 accession. Following pollination of a third accession, Col-0, with pollen from the distinct fathers resulted in the recovery of two viable herbicide resistant plants, TP³ 1 and TP³ 2; see FIG. 4 (A). The plants passed the previously introduced tests for triparental plants: They segregated an active YFP, and exhibited a triploid profile in the flow cytometry assay; see FIGS. 4 (B) and (C). To test, whether, in fact, the chromosomes of three different accessions had been transmitted, accession specific restriction fragment length polymorphisms (RFLP) on each of the five chromosomes have been targeted. For this approach, primers flanking Col-0, C24, and Ler characteristic RFLPs on all five chromosome were designed. RFLPs were chosen on the basis of the POLYMORPH webtool (Clark et al., Science 317 (2007), 338-342; Zeller et al., Genome research 18 (2008), 918-929) and are listed in the following Table. Notably, polymorphisms characteristic for each of the three accessions on all chromosomes have been detected (FIG. 4 (D)), indicating that all three genomes have been inherited.

| Primer Name | Sequence of Primers 5′-3′ (SEQ ID NO) | RE | Chromosome |
|---|---|---|---|
| DT51_Fw | CAGGACATCCACCTAGAAACAAGAAGAC | AluI | Chr. 1 |
| | (SEQ ID NO: 18) | | |
| DT52_Rev | TGAATGTGTGCAGATCATGGAGAAGGAC | | |
| | (SEQ ID NO: 19) | | |
| DT53_Fw | TGTACCTGGATTCGATAGAACTGC | NdeI | |
| | (SEQ ID NO: 20) | | |
| DT54_Rev | AGAAGTCCAAGCATGTCCTATGGT | | |
| | (SEQ ID NO: 21) | | |
| DT55_Fw | TAAGATTGCTCAGAAGCTGAGTGAG | SacI | |
| | (SEQ ID NO: 22) | | |
| DT56_Rev | CGACACACAATGTATCTGTTTACCC | | |
| | (SEQ ID NO: 23) | | |
| DT92_Fw | ACCTCTAAACGTGATTGCAACTGTGAG | HindIII | Chr. 2 |
| | (SEQ ID NO: 24) | | |
| DT93_Rev | TGTGCCAACAATCTCTCATGTACATGG | | |
| | (SEQ ID NO: 25) | | |
| DT59_Fw | TGGTCGTTGCTTAAGTTGTTAAGCTCG | PstI | |
| | (SEQ ID NO: 26) | | |
| DT60_Rev | CAATACAAGACACTTTCTCCAATATGGG | | |
| | (SEQ ID NO: 27) | | |
| DT61_Fw | TGGTTCACATGTACGCCAAGTGTTACC | BamHI | |
| | (SEQ ID NO: 28) | | |
| DT89_Rev | CTTCCCTTCATTAACCATCCCTGTGTG | | |
| | (SEQ ID NO: 29) | | |
| DT116_Fw | TTATTTCCGACTTTGTCTTTGCCATCG | SacI | Chr. 3 |
| | (SEQ ID NO: 30) | | |
| DT117_Rev | TGCTAGATTATGCCTTTGATCACAAGC | | |
| | (SEQ ID NO: 31) | | |
| DT65_Fw | GCTGCAATGGTAAACAAGGAGGAAGAG | HindIII | |
| | (SEQ ID NO: 32) | | |
| DT66_Rev | CGGGATCATAATGCCGATATTCTTCGG | | |
| | (SEQ ID NO: 33) | | |
| DT67_Fw | TGTCTTCAGCATATATGCCGATTGGAG | HindIII | |
| | (SEQ ID NO: 34) | | |
| DT68_Rev | CAATCTCTTCAGGTGAGATAATGAGCG | | |
| | (SEQ ID NO: 35) | | |
| DT96_Fw | ACACGCAAGAGAGAATTGGATGATACC | ClaI | Chr. 4 |
| | (SEQ ID NO: 36) | | |
| DT97_Rev | AGCAGAATCTCAATCTCGATGGTTGTG | | |
| | (SEQ ID NO: 37) | | |
| DT71_Fw | TCTCTTGCTCTCTTCTCTCTCACACAG | EcoRV | |
| | (SEQ ID NO: 38) | | |
| DT72_Rev | ACCTCTTGTGGAGTATCAAGCCAAGTG | | |
| | (SEQ ID NO: 39) | | |
| DT98_Fw | TACAGAGTCCAAGTCGTTGTCATGCTC | PstI | |
| | (SEQ ID NO: 40) | | |
| DT99_Rev | TGCGTGACTGCTATATCCTAAAGGAGG | | |
| | (SEQ ID NO: 41) | | |
| DT77_Fw | CAACGAGAAGTTGGATTGTGAGGTG | SpeI | Chr. 5 |
| | (SEQ ID NO: 42) | | |
| DT78_Rev | GGATGAAGGAGCATATGGAGATAGC | | |
| | (SEQ ID NO: 43) | | |
| DT79_Fw | GTATTCACGACCATCGTAGCTGTCCAC | SmaI | |
| | (SEQ ID NO: 44) | | |
| DT80_Rev | AACTCGCCTCTGAAGCTGGTCAATGAG | | |
| | (SEQ ID NO: 45) | | |
| DT81_Fw | AACAGTAATGGTCCCCATTCACGGTGG | HindIII | |
| | (SEQ ID NO: 46) | | |
| DT82_Rev | CCAATTAGACCTTGAAGGAGCAGGACC | | |
| | (SEQ ID NO: 47) | | |

1. A polyploid plant obtainable by crossing of one female parent plant with at least two male parent plants.

2. The polyploid plant of claim 1, which is a triparental plant.

3. The polyploid plant of claim 1, wherein the at least two male parent plants are genetically distinct, preferably wherein they belong to different cultivars or varieties.

4. The polyploid plant of claim 1, wherein at least one male parent plant is normally incompatible with the female parent plant.

5. The polyploid plant of claim 1, which is a monocotyledonous or dicotyledonous plant.

6. A population of polyploid plants of claim 1.

7. An organ, part, tissue, cell, seed or offspring of the plant of claim 1.

8. A method for obtaining a polyploid plant of claim 1 comprising
(a) providing at least three parental plants, wherein at least one first and one second male parent plant comprises a component which provides a selectable marker;
(b) crossing the at least three parental plants to provide offspring;
(c) selecting from the offspring from step (b) based on the presence of the marker; and optionally
(d) generating from the offspring selected in step (c) marker-free polyploid plants by
(i) crossing or selfing at least two of the offspring selected in step (c) to produce further offspring; and
(ii) selecting plants from the further offspring obtained in step (d)(i) based on the absence of the marker so as to obtain the polyploid plant.

9. The method of claim 8, wherein the selectable marker confers herbicide, antibiotic and/or antimetabolite resistance and/or a detectable, preferably visible output, preferably wherein the selectable marker is preferably selected based on a herbicide resistance gene selected from the group consisting of the bar gene and the pat gene, and/or an antibiotic resistance gene selected from the group consisting of the nptII gene, the hpt gene, the acc3 gene and the aadA gene, and/or a gene conferring antimetabolite resistance such as the dhfr gene, and/or a bioreporter gene selected from the group consisting of a GFP gene, a YFP gene, a CFP gene, a BFP gene, a Venus gene, a dsRED gene, a hcRED gene, a mCherry gene, a mPlum gene, a mOrange gene, a zoanFP gene, a Cerulan gene, a luc gene, a cat gene, lacZ gene and uidA gene as well as modified and/or enhanced versions thereof.

10. The method of claim 8, wherein the at least one first and one second male parent plant are homozygous for the component which provides the selectable male.

11. A polyploid plant obtainable by the method of claim 8 or a part, tissue, cell, seed or offspring of such a polyploid plant.

12. The method of claim 8, wherein at least one parental plant comprises a transgenic plant or progeny of a transgenic plant, wherein the transgenic plant comprising
(i) a stably integrated expression cassette, said expression cassette comprising a nucleotide sequence encoding a promoter, preferably a constitutive promoter which is operably linked to a nucleotide sequence encoding a transactivator capable of regulating a target nucleotide sequence, or
(ii) a stably integrated first expression cassette comprising a nucleotide sequence encoding a promoter, preferably a constitutive promoter, operably linked to a nucleotide sequence encoding a first selectable marker, preferably a male which provides antibiotic, herbicide and/or antimetabolite resistance and/or reporter gene expression when expressed.

13. The method of claim 8, wherein at least one parental plant comprises a transgenic plant comprising
(i) a stably integrated expression cassette said expression cassette comprising a target nucleic acid sequence capable of being activated by a transactivator, operably linked to a nucleotide sequence which encodes a selectable marker, preferably a marker which provides antibiotic, herbicide and/or antimetabolite resistance and/or reporter gene expression when expressed, or
(ii) a stably integrated second expression cassette comprising a nucleotide sequence encoding a promoter, preferably a constitutive promoter operably linked to a nucleotide sequence encoding a second selectable marker, which provides antibiotic, herbicide and/or antimetabolite resistance and/or reporter gene expression when expressed.

14. A population of polyploid plants comprising at least three parental plants of claim 8, preferably at least one transgenic plant and at least one non-transgenic plant, wherein growing the population under suitable conditions allows pollination of said non-transgenic plant as the female plant by pollen of a transgenic plant as the male plant.

15. A transgenic tri-parental plant and/or progeny of such a plant, which comprises
(i) a stably integrated first expression cassette comprising a nucleotide sequence encoding a promoter, preferably a constitutive promoter operably linked to a nucleotide sequence encoding a transactivator and a second expression cassette comprising a target nucleotide sequence, capable of being activated by said transactivator polypeptide operably linked to a nucleotide sequence which encodes a selectable marker, or
(ii) a stably integrated first expression cassette comprising a nucleotide sequence encoding a promoter, preferably a constitutive promoter operably linked to a nucleotide sequence encoding a first selectable marker and a second expression cassette comprising a nucleotide sequence encoding a promoter, preferably a constitutive promoter, operably linked to a nucleotide sequence encoding a second selectable marker, wherein the selectable marker provides antibiotic, herbicide and/or antimetabolite resistance when expressed.

16. The polyploid plant of claim 2, wherein the at least two male parent plants are genetically distinct, preferably wherein they belong to different cultivars or varieties.

17. The polyploid plant of claim 2, wherein at least one male parent plant is normally incompatible with the female parent plant.

18. The polyploid plant of claim 3, wherein at least one male parent plant is normally incompatible with the female parent plant.

19. The polyploid plant of claim 2, which is a monocotyledonous or dicotyledonous plant.

20. A population of polyploid plants comprising at least three parental plants of claim 9, preferably at least one transgenic plant and at least one non-transgenic plant, wherein growing the population under suitable conditions allows pollination of said non-transgenic plant as the female plant by pollen of a transgenic plant as the male plant.
